(19) United States
(12) Patent Application Publication
(10) Pub. No.: US 2023/0130619 A1
(43) Pub. Date: Apr. 27, 2023

(54) MACHINE LEARNING PIPELINE USING DNA-ENCODED LIBRARY SELECTIONS

(71) Applicant: Insitro, Inc., South San Francisco, CA (US)

(72) Inventors: Ralph Ma, South San Francisco, CA (US); Gabriel Dreiman, South San Francisco, CA (US); Fiorella Ruggiu, South San Francisco, CA (US); Adam Riesselman, Brisbane, CA (US); Bowen Liu, South San Francisco, CA (US); Mohammad Muneeb Sultan, South San Francisco, CA (US)

(21) Appl. No.: 17/971,366

(22) Filed: Oct. 21, 2022

Related U.S. Application Data
(60) Provisional application No. 63/271,029, filed on Oct. 22, 2021.

(51) Int. Cl. G16B 15/30; G06F 18/27

(52) U.S. Cl. CPC G16B 15/30; G06F 18/27

(57) ABSTRACT

Embodiments of the disclosure involve training machine learned models using DNA-encoded library experimental data outputs and for deploying the trained machine learned models for conducting a virtual compound screen, for performing a hit selection and analysis, or for predicting binding affinities between compounds and targets. Machine learned models are trained using one or more augmentations that selectively expand molecular representations of a training dataset. Furthermore, machine learned models are trained to account for confounding covariates, thereby improving the machine learned models' abilities to conduct a virtual screen, perform a hit selection, and to predict binding affinities.

### CROSS-REFERENCE TO RELATED APPLICATIONS

This application claims the benefit of and priority to U.S. Provisional Patent Application No. 63/271,029 filed Oct. 22, 2021, the entire disclosure of which is hereby incorporated by reference in its entirety for all purposes.

### BACKGROUND OF THE INVENTION

Small molecule drug discovery begins with the identification of putative chemical matter that binds to targets of interest. This can be achieved with experimental techniques such as high throughput screening or in silico methodologies such as docking and generative modeling. DNA encoded library (DEL) screening is a high throughput experimental technique used to screen diverse sets of chemical matter against targets of interest to identify binders.

DELs are DNA barcode-labeled pooled compound collections that are incubated with an immobilized protein target in a process referred to as panning. The mixture is then washed to remove non-binders, and the remaining bound compounds are eluted, amplified, and sequenced to identify putative binders. DELs provide a quantitative readout for up to hundreds of millions of compounds. However, conventional DEL experiments yield datasets with low signal-to-noise ratio. Specifically, DEL readouts can contain substantial experimental noise and biases caused by sources including DEL members binding the protein immobilization media or differences in starting population (load). When machine learning models are trained on data derived from DEL experiments, the noise and biases often contribute towards the poor performance of these models. Thus, there is a need for improved methodologies for handling DEL experimental outputs to build improved machine learning models.

### SUMMARY

Disclosed herein are methods, non-transitory computer readable media, and systems for training machine learned models using DEL experimental datasets and for deploying the trained machine learned models for conducting virtual compound screens, for performing hit selection and analyses, or for predicting binding affinities between compounds and targets. Conducting a virtual compound screen enables identifying compounds from a library (e.g., virtual library) that are likely to bind to a target, such as a protein target. Performing a hit selection enables identification of compounds that likely exhibit a desired activity. For example, a hit can be a compound that binds to a target (e.g., a protein target) and therefore, exhibits a desired effect by binding to the target. Predicting binding affinity between compounds and targets can result in the identification of compounds that exhibit a desired binding affinity. For example, binding affinity values can be continuous values and therefore, can be indicative of different types of binders (e.g., strong binder or weak binder). This enables the identification and categorization of compounds that exhibit different binding affinities to targets.

In various embodiments, the machine learned models disclosed herein include one or both of a classification model and a regression model. In various embodiments, the classification model is trained using one or more augmentations that selectively expand molecular representations of a training dataset. In various embodiments, the regression model is trained to model DEL sequencing counts, accounting for two or more confounding sources of noise and biases, hereafter referred to as covariates. Thus, the machine learned models disclosed herein generate predictions having improved accuracy when conducting virtual compound screens, performing hit selection and analyses, or predicting binding affinities between compounds and targets.

Additionally disclosed herein is a method for conducting a molecular screen for a target, the method comprising: obtaining a plurality of compounds from a library; for each of one or more of the plurality of compounds: applying the compound as input to one or both of: (A) a classification model for predicting candidate compounds likely to bind to the target, wherein the classification model is trained using one or more augmentations that selectively expand molecular representations of a training dataset used to train the classification model; and (B) a regression model trained to predict a value indicative of binding affinity between compounds and targets, wherein the regression model is trained using compounds with corresponding DNA-encoded library (DEL) outputs to incorporate two or more covariates for predicting the value indicative of binding affinity; and selecting candidate compounds as predicted binders of the target based on one or both of the outputs of the classification model and the regression model. In various embodiments, the molecular screen is a virtual molecular screen. In various embodiments, the library is a virtual library. In various embodiments, the library is a physical library.

Additionally disclosed herein is a method for conducting a hit selection, the method comprising: obtaining a compound; applying the compound as input to one or both of: (A) a classification model for predicting candidate compounds likely to bind to targets, wherein the classification model is trained using one or more augmentations that selectively expand molecular representations of a training dataset used to train the classification model; and (B) a regression model trained to predict a value indicative of binding affinity between compounds and targets, wherein the regression model is trained using compounds with corresponding DNA-encoded library (DEL) outputs to incorporate two or more covariates for predicting the value indicative of binding affinity; and selecting candidate compounds as predicted binders of the target based on one or both of the outputs of the classification model and the regression model. In various embodiments, applying the compound as input comprises applying the compound as input to both the classification model and the regression model. In various embodiments, methods disclosed herein further comprise: identifying overlapping candidate compounds predicted by the classification model and by the regression model based on the value indicative of binding affinity; and selecting a subset of the overlapping candidate compounds as predicted binders of the target.

In various embodiments, applying the compound as input comprises applying the compound as input to two or more classification models. In various embodiments, methods disclosed herein further comprise: identifying overlapping candidate compounds predicted by the two or more classification models; and selecting a subset of the overlapping candidate compounds as predicted binders of the target. In various embodiments, applying the compound as input comprises applying the compound as input to two or more regression models. In various embodiments, applying the compound as input comprises applying the compound as input to three regression models. In various embodiments, methods disclosed herein further comprise: identifying overlapping candidate compounds predicted by the two or more regression models; and selecting a subset of the overlapping candidate compounds as predicted binders of the target. In various embodiments, the classification model is a neural network. In various embodiments, the classification model is a graph neural network. In various embodiments, the classification model is a GIN-E model with an enabled virtual node. In various embodiments, the classification model terminates in a layer that maps a graph tensor into an embedding. In various embodiments, the classification model predicts a binary value indicating whether candidate compounds are likely to bind to the target. In various embodiments, the classification model predicts multi-class values indicating whether candidate compounds are likely to bind to the target. In various embodiments, the multi-class values include any of a strong binder, a weak binder, a non-binder, and an off target binder.

In various embodiments, applying the compound as input to a classification model for predicting candidate compounds likely to bind to the target comprises: determining one of distance or clustering of one or more compounds within the embedding; based on the distance or clustering of the one or more compounds within the embedding, determining whether to label the one or more compounds as candidate compounds. In various embodiments, the classification model is trained using a loss function. In various embodiments, the loss function is any one of a binary cross entropy loss, focal loss, arc loss, cosface loss, cosine based loss, or loss function based on a BEDROC metric.

In various embodiments, the classification model is trained using pre-selected labels. In various embodiments, the pre-selected labels are selected by: evaluating a plurality of labels by testing performance of label prediction models trained using subsets of labels from the plurality of labels. In various embodiments, evaluating the plurality of labels by testing performance of label prediction models using subsets of labels comprises: for each subset of labels: training a label prediction model to predict the subset of labels based on molecular data; and validating the label prediction model using a validation dataset to determine one or more metrics for evaluating the subset of labels; selecting one or more of the subset of labels as the pre-selected labels based on the one or more metrics of the subset of labels. In various embodiments, training a label prediction model to predict the subset of labels based on molecular data comprises: converting structure formats into molecular representations; providing the molecular representations as input to the label prediction model to predict the subset of labels. In various embodiments, the structure formats are any one of simplified molecular-input line-entry system (SMILES) string, MDL Molfile (MDL MOL), Structure Data File (SDF), Protein Data Bank (PDB), Molecule specification file (xyz), International Union of Pure and Applied Chemistry (IUPAC) International Chemical Identifier (InChI), and Tripos Mol2 file (mol2) format. In various embodiments, the molecular representations are any one of molecular fingerprints or molecular graphs. In various embodiments, the label prediction models are any one of a regression model, classification model, random forest model, decision tree, support vector machine, Naïve Bayes model, clustering model (e.g., k-means cluster), or neural network.

In various embodiments, the classification model is trained by: for one or more training epochs, determining a loss value; and updating parameters of the classification model using the determined loss values across the one or more training epochs. In various embodiments, the classification model is further trained by: evaluating the performance of the classification model based on a metric. In various embodiments, the metric is one or more of a Boltzmann-Enhanced Discrimination of Receiver Operating Curve (BEDROC) metric, an Area Under ROC (AUROC) metric, and an average precision (AVG-PRC) metric.

In various embodiments, the one or more augmentations used selectively to expand molecular representations of a training dataset comprise: enumerating tautomers of compounds during training, performing a transformation of one or more compounds, wherein the transformation is any one of matched molecular pair transforms or bioisosteres, Bemis-Murcko scaffolds, node dropout, or edge dropout, generating a representation of ionization states, generating mixtures of structures associated with a tag, mixtures of tautomers, mixtures of conformers, mixtures of ionization states, or mixtures of transformations of the one or more compounds, or generating conformers. In various embodiments, the tag associated with mixtures of structures is a DNA sequence.

In various embodiments, the classification model comprises a tunable hyperparameter that controls implementation of the one or more augmentations. In various embodiments, the tunable hyperparameter is a probability value that controls the implementation of the one or more augmentations. In various embodiments, the one or more augmentations are further selected for implementation using a random number generator. In various embodiments, the classification model or the regression model are trained using a training set, and validated using a validation set. In various embodiments, the training set comprises one or more DEL libraries, and wherein the validation set comprises one or more different DEL libraries. In various embodiments, the training set and validation set are split from a full dataset to improve generalization of the classification model or the regression model. In various embodiments, the training set and validation set are split from a full dataset by: generating a representative sample of compounds of the DEL by ensuring each building block in the DEL synthesis appears at least once in the representative sample, wherein the compounds are each composed of one or more building blocks; generating molecular fingerprints of the compounds in the representative sample; assigning the compounds to a plurality of groups by clustering the molecular fingerprints of the compounds; and assigning a first subset of the plurality of groups to the training set and assigning a second subset of the plurality of groups to the validation set. In various embodiments, the training set and validation set are further split by: prior to assigning the first subset of the plurality of groups and assigning the second subset of the plurality of groups, supplementing the plurality of groups by further clustering molecular fingerprints of compounds that were not included in the representative sample of compounds of the DEL. In various embodiments, further clustering molecular fingerprints of compounds that were not included in the representative sample of compounds of the DEL comprises: determining distances between molecular fingerprints of compounds not included in the representative sample to one or more compounds in the clusters formed by the representative sample of compounds of the DEL; and assigning compounds not included in the representative sample to the clusters based on the determined distances. In various embodiments, the clustering comprises hierarchical clustering.

Additionally disclosed herein is a method for predicting binding affinity between a compound and a target, the method comprising: obtaining the compound; applying the compound as input to a regression model trained to predict a value indicative of binding affinity between compounds and targets, wherein the regression model is trained using compounds with corresponding DNA-encoded library (DEL) outputs to incorporate two or more covariates for predicting the value indicative of binding affinity. In various embodiments, the regression model is further trained using one or more augmentations that selectively expand molecular representations of a training dataset used to train the regression model. In various embodiments, the one or more augmentations comprise: enumerating tautomers of compounds during training, performing a transformation of one or more compounds, wherein the transformation is any one of matched molecular pair transforms or bioisosteres, Bemis-Murcko scaffolds, node dropout, or edge dropout, generating a representation of protomers (formal charges states), generating mixtures of structures associated with a tag, mixtures of tautomers, mixtures of conformers, mixtures of promoters, or mixtures of transformations of the one or more compounds, or generating conformers. In various embodiments, the tag associated with mixtures of structures is a DNA sequence.

In various embodiments, the regression model comprises a tunable hyperparameter that controls implementation of the one or more augmentations. In various embodiments, the tunable hyperparameter is a probability value that controls the implementation of the one or more augmentations. In various embodiments, the one or more augmentations are further selected for implementation using a random number generator. In various embodiments, the regression model comprises a first portion that analyzes the compound and outputs a fixed dimensional embedding.

In various embodiments, applying the compound as input to the regression model trained to predict a value indicative of binding affinity comprises: using the embedding to generate an enrichment value representing the value indicative of binding affinity. In various embodiments, using the embedding to generate the enrichment value comprises providing the embedding as input to a feed forward network, wherein the feed forward network generates the enrichment value for a modeled experiment. In various embodiments, the enrichment value represents an intermediate value within the regression model. In various embodiments, the regression model is further trained to predict one or more DEL predictions that model one or more experiments, wherein at least one of the one or more DEL predictions is generated using at least the intermediate value of the enrichment value. In various embodiments, applying the compound as input to the regression model trained to predict a value indicative of binding affinity further comprises: using the embedding to generate one or more covariate enrichment values that correspond to one or more negative control experiments.

In various embodiments, the negative control experiment models effects of the covariate across a set of proteins. In various embodiments, the negative control experiment models effects of the covariate for a binding site. In various embodiments, the binding site is a target binding site or an orthogonal binding site. In various embodiments, each of the two or more covariates are any of non-specific binding via controls and other targets data, starting tag imbalance, experimental conditions, chemical reaction yields, side and truncated products, errors from the library synthesis, DNA affinity to target, sequencing depth, and sequencing noise such as PCR bias.

In various embodiments, the regression model is trained by: back-propagating an error between predicted DEL outputs and observed experimental DEL outputs using a gradient based optimization technique to minimize a loss function. In various embodiments, a first of the predicted DEL outputs is derived from a target enrichment value, and wherein at least a second of the predicted DEL outputs is derived from a covariate enrichment value. In various embodiments, the first of the predicted DEL outputs is derived by combining at least the target enrichment value and the covariate enrichment value. In various embodiments, the target enrichment value and the covariate enrichment value are combined using parameters of the regression model, wherein the parameters of the regression model are adjusted to minimize the loss function. In various embodiments, the loss function is any one of a mean square error, log likelihood of a negative binomial distribution, zero inflated negative binomial, or log likelihood of a Poisson distribution.

In various embodiments, the first portion of the regression model is an encoding network. In various embodiments, the encoding network is any one of a graph neural network, attention based model, a multilayer perceptron. In various embodiments, the first portion of the regression model is not a trainable network. In various embodiments, the DEL outputs comprise one or more of DEL counts, DEL reads, or DEL indices. In various embodiments, the value indicative of binding affinity between compounds and targets is one or more of DEL counts, DEL reads, or DEL indices.

In various embodiments, the value indicative of binding affinity between compounds and targets represents a denoised and/or debiased DEL count, DEL read, or DEL index that is absent effects of the one or more covariates. In various embodiments, the target is a binding site. In various embodiments, the target is a protein binding site. In various embodiments, the target is a protein-protein interaction interface.

Additionally disclosed herein is a non-transitory computer readable medium for conducting a molecular screen for a target, the non-transitory computer readable medium comprising instructions that, when executed by a processor, cause the processor to: obtain a plurality of compounds from a library; for each of one or more of the plurality of compounds: apply the compound as input to one or both of: (A) a classification model for predicting candidate compounds likely to bind to the target, wherein the classification model is trained using one or more augmentations that selectively expand molecular representations of a training dataset used to train the classification model; and (B) a regression model trained to predict a value indicative of binding affinity between compounds and targets, wherein the regression model is trained using compounds with corresponding DNA-encoded library (DEL) outputs to incorporate two or more covariates for predicting the value indicative of binding affinity; and select candidate compounds as predicted binders of the target based on one or both of the outputs of the classification model and the regression model.

In various embodiments, the molecular screen is a virtual molecular screen. In various embodiments, the library is a virtual library. In various embodiments, the library is a physical library.

Additionally disclosed herein is a non-transitory computer readable medium for conducting a hit selection, the non-transitory computer readable medium comprising instructions that, when executed by a processor, cause the processor to: obtain a compound; apply the compound as input to one or both of: (A) a classification model for predicting candidate compounds likely to bind to targets, wherein the classification model is trained using one or more augmentations that selectively expand molecular representations of a training dataset used to train the classification model; and (B) a regression model trained to predict a value indicative of binding affinity between compounds and targets, wherein the regression model is trained using compounds with corresponding DNA-encoded library (DEL) outputs to incorporate two or more covariates for predicting the value indicative of binding affinity; and select candidate compounds as predicted binders of the target based on one or both of the outputs of the classification model and the regression model. In various embodiments, applying the compound as input comprises applying the compound as input to both the classification model and the regression model. In various embodiments, non-transitory computer readable media disclosed herein further comprise instructions that, when executed by the processor, cause the processor to: identify overlapping candidate compounds predicted by the classification model and by the regression model based on the value indicative of binding affinity; and select a subset of the overlapping candidate compounds as predicted binders of the target.

In various embodiments, the instructions that cause the processor to apply the compound as input further comprise instructions that, when executed by the processor, cause the processor to apply the compound as input to two or more classification models. In various embodiments, non-transitory computer readable media disclosed herein further comprise instructions that, when executed by a processor, cause the processor to: identify overlapping candidate compounds predicted by the two or more classification models; and select a subset of the overlapping candidate compounds as predicted binders of the target. In various embodiments, the instructions that cause the processor to apply the compound as input further comprise instructions that, when executed by the processor, cause the processor to apply the compound as input to two or more regression models. In various embodiments, the instructions that cause the processor to apply the compound as input further comprise instructions that, when executed by the processor, cause the processor to apply the compound as input to three regression models. In various embodiments, a non-transitory computer readable media disclosed herein further comprise instructions that, when executed by a processor, cause the processor to: identify overlapping candidate compounds predicted by the two or more regression models; and select a subset of the overlapping candidate compounds as predicted binders of the target.

In various embodiments, the classification model is a neural network. In various embodiments, the classification model is a graph neural network. In various embodiments, the classification model is a GIN-E model with an enabled virtual node. In various embodiments, the classification model terminates in a layer that maps a graph tensor into an embedding. In various embodiments, the classification model predicts a binary value indicating whether candidate compounds are likely to bind to the target. In various embodiments, the classification model predicts multi-class values indicating whether candidate compounds are likely to bind to the target. In various embodiments, the multi-class values include any of a strong binder, a weak binder, a non-binder, and an off target binder. In various embodiments, the instructions that cause the processor to apply the compound as input to a classification model for predicting candidate compounds likely to bind to the target further comprises instructions that, when executed by a processor, cause the processor to: determine one of distance or clustering of one or more compounds within the embedding; and based on the distance or clustering of the one or more compounds within the embedding, determine whether to label the one or more compounds as candidate compounds. In various embodiments, the classification model is trained using a loss function. In various embodiments, the loss function is any one of a binary cross entropy loss, focal loss, arc loss, cosface loss, cosine based loss, or loss function based on a BEDROC metric. In various embodiments, the classification model is trained using pre-selected labels. In various embodiments, the pre-selected labels are selected by executing instructions that cause the processor to: evaluate a plurality of labels by testing performance of label prediction models trained using subsets of labels from the plurality of labels. In various embodiments, the instructions that cause the processor to evaluate the plurality of labels by testing performance of label prediction models using subsets of labels further comprise instructions that, when executed by a processor, cause the processor to: for each subset of labels: train a label prediction model to predict the subset of labels based on molecular data; and validate the label prediction model using a validation dataset to determine one or more metrics for evaluating the subset of labels; and select one or more of the subset of labels as the pre-selected labels based on the one or more metrics of the subset of labels.

In various embodiments, the instructions that cause the processor to train a label prediction model to predict the subset of labels based on molecular data further comprise instructions that, when executed by a processor, cause the processor to: convert structure formats into molecular representations; provide the molecular representations as input to the label prediction model to predict the subset of labels. In various embodiments, the structure formats are any one of simplified molecular-input line-entry system (SMILES) string, MDL Molfile (MDL MOL), Structure Data File (SDF), Protein Data Bank (PDB), Molecule specification file (xyz), International Union of Pure and Applied Chemistry (IUPAC) International Chemical Identifier (InChI), and Tripos Mol2 file (mol2) format. In various embodiments, the molecular representations are any one of molecular fingerprints or molecular graphs. In various embodiments, the label prediction models are any one of a regression model, classification model, random forest model, decision tree, support vector machine, Naïve Bayes model, clustering model (e.g., k-means cluster), or neural network.

In various embodiments, the classification model is trained by: for one or more training epochs, determining a loss value; and updating parameters of the classification model using the determined loss values across the one or more training epochs. In various embodiments, the classification model is further trained by: evaluating the performance of the classification model based on a metric. In various embodiments, the metric is one or more of a Boltzmann-Enhanced Discrimination of Receiver Operating Curve (BEDROC) metric, an Area Under ROC (AUROC) metric, and an average precision (AVG-PRC) metric.

In various embodiments, the one or more augmentations comprise: enumerating tautomers of compounds during training, performing a transformation of one or more compounds, wherein the transformation is any one of matched molecular pair transforms or bioisosteres, Bemis-Murcko scaffolds, node dropout, or edge dropout, generating a representation of ionization states, generating mixtures of structures associated with a tag, mixtures of tautomers, mixtures of conformers, mixtures of ionization states, or mixtures of transformations of the one or more compounds, or generating conformers. In various embodiments, the tag associated with mixtures of structures is a DNA sequence.

In various embodiments, the classification model comprises a tunable hyperparameter that controls implementation of the one or more augmentations. In various embodiments, the tunable hyperparameter is a probability value that controls the implementation of the one or more augmentations. In various embodiments, the one or more augmentations are further selected for implementation using a random number generator.

In various embodiments, the classification model or the regression model are trained using a training set, and validated using a validation set. In various embodiments, the training set comprises one or more DEL libraries, and wherein the validation set comprises one or more different DEL libraries. In various embodiments, the training set and validation set are split from a full dataset to improve generalization of the classification model or the regression model. In various embodiments, the training set and validation set are split from a full dataset by executing instructions that cause the processor to: generate a representative sample of compounds of the DEL by ensuring each building block in the DEL synthesis appears at least once in the representative sample, wherein the compounds are each composed of one or more building blocks; generate molecular fingerprints of the compounds in the representative sample; assign the compounds to a plurality of groups by clustering the molecular fingerprints of the compounds; and assign a first subset of the plurality of groups to the training set and assigning a second subset of the plurality of groups to the validation set. In various embodiments, the training set and validation set are further split by: prior to assigning the first subset of the plurality of groups and assigning the second subset of the plurality of groups, supplementing the plurality of groups by further clustering molecular fingerprints of compounds that were not included in the representative sample of compounds of the DEL. In various embodiments, the instructions that cause the processor to further cluster molecular fingerprints of compounds that were not included in the representative sample of compounds of the DEL further comprise instructions that, when executed by the processor, cause the processor to: determine distances between molecular fingerprints of compounds not included in the representative sample to one or more compounds in the clusters formed by the representative sample of compounds of the DEL; and assign compounds not included in the representative sample to the clusters based on the determined distances. In various embodiments, the clustering comprises hierarchical clustering.

Additionally disclosed herein is a non-transitory computer readable medium for predicting binding affinity between a compound and a target, the non-transitory computer readable medium comprising instructions that, when executed by a processor, cause the processor to: obtain the compound; apply the compound as input to a regression model trained to predict a value indicative of binding affinity between compounds and targets, wherein the regression model is trained using compounds with corresponding DNA-encoded library (DEL) outputs to incorporate two or more covariates for predicting the value indicative of binding affinity. In various embodiments, the regression model is further trained using one or more augmentations that selectively expand molecular representations of a training dataset used to train the regression model. In various embodiments, the one or more augmentations comprise: enumerating tautomers of compounds during training, performing a transformation of one or more compounds, wherein the transformation is any one of matched molecular pair transforms or bioisosteres, Bemis-Murcko scaffolds, node dropout, or edge dropout, generating a representation of protomers (formal charges states), generating mixtures of structures associated with a tag, mixtures of tautomers, mixtures of conformers, mixtures of promoters, or mixtures of transformations of the one or more compounds, or generating conformers. In various embodiments, the tag associated with mixtures of structures is a DNA sequence. In various embodiments, the regression model comprises a tunable hyperparameter that controls implementation of the one or more augmentations. In various embodiments, the tunable hyperparameter is a probability value that controls the implementation of the one or more augmentations. In various embodiments, the one or more augmentations are further selected for implementation using a random number generator. In various embodiments, the regression model comprises a first portion that analyzes the compound and outputs a fixed dimensional embedding. In various embodiments, the instructions that cause the processor to apply the compound as input to the regression model trained to predict a value indicative of binding affinity further comprises instructions that, when executed by the processor, cause the processor to: use the embedding to generate an enrichment value representing the value indicative of binding affinity.

In various embodiments, the instructions that cause the processor to use the embedding to generate the enrichment value further comprises instructions that, when executed by the processor, cause the processor to provide the embedding as input to a feed forward network, wherein the feed forward network generates the enrichment value for a modeled experiment. In various embodiments, the enrichment value represents an intermediate value within the regression model. In various embodiments, the regression model is further trained to predict one or more DEL predictions that model one or more experiments, wherein at least one of the one or more DEL predictions is generated using at least the intermediate value of the enrichment value. In various embodiments, the instructions that cause the processor to applying the compound as input to the regression model trained to predict a value indicative of binding affinity further comprises instructions that, when executed by the processor, cause the processor to: use the embedding to generate one or more covariate enrichment values that correspond to one or more negative control experiments. In various embodiments, the negative control experiment models effects of the covariate across a set of proteins. In various embodiments, the negative control experiment models effects of the covariate for a binding site. In various embodiments, the binding site is a target binding site or an orthogonal binding site. In various embodiments, each of the two or more covariates are any of non-specific binding via controls and other targets data, starting tag imbalance, experimental conditions, chemical reaction yields, side and truncated products, errors from the library synthesis, DNA affinity to target, sequencing depth, and sequencing noise such as PCR bias.

In various embodiments, the regression model is trained by: back-propagating an error between predicted DEL outputs and observed experimental DEL outputs using a gradient based optimization technique to minimize a loss function. In various embodiments, a first of the predicted DEL outputs is derived from a target enrichment value, and wherein at least a second of the predicted DEL outputs is derived from a covariate enrichment value. In various embodiments, the first of the predicted DEL outputs is derived by combining at least the target enrichment value and the covariate enrichment value. In various embodiments, the target enrichment value and the covariate enrichment value are combined using parameters of the regression model, wherein the parameters of the regression model are adjusted to minimize the loss function. In various embodiments, the loss function is any one of a mean square error, log likelihood of a negative binomial distribution, zero inflated negative binomial, or log likelihood of a Poisson distribution.

In various embodiments, the first portion of the regression model is an encoding network. In various embodiments, the encoding network is any one of a graph neural network, attention based model, a multilayer perceptron. In various embodiments, the first portion of the regression model is not a trainable network. In various embodiments, the DEL outputs comprise one or more of DEL counts, DEL reads, or DEL indices. In various embodiments, the value indicative of binding affinity between compounds and targets is one or more of DEL counts, DEL reads, or DEL indices. In various embodiments, the value indicative of binding affinity between compounds and targets represents a denoised and/or debiased DEL count, DEL read, or DEL index that is absent effects of the one or more covariates. In various embodiments, the target is a binding site. In various embodiments, the target is a protein binding site. In various embodiments, the target is a protein-protein interaction interface.

### BRIEF DESCRIPTION OF THE DRAWINGS

These and other features, aspects, and advantages of the present invention will become better understood with regard to the following description and accompanying drawings. It is noted that, wherever practicable, similar or like reference numbers may be used in the figures and may indicate similar or like functionality. For example, a letter after a reference numeral, such as “DEL experiment 115A,” indicates that the text refers specifically to the element having that particular reference numeral. A reference numeral in the text without a following letter, such as “DEL experiment 115,” refers to any or all of the elements in the figures bearing that reference numeral (e.g. “DEL experiment 115” in the text refers to reference numerals “DEL experiment 115A” and/or “DEL experiment 115B” in the figures).

FIG. 1A depicts an example system environment involving a compound analysis system, in accordance with an embodiment.

FIG. 1B depicts a block diagram of a compound analysis system, in accordance with an embodiment.

FIG. 2 depicts a flow diagram for splitting and/or labeling a DNA encoded library (DEL) dataset for training a regression model or a classification model, in accordance with an embodiment.

FIG. 3A depicts a flow process for dataset splitting, in accordance with an embodiment.

FIG. 3B depicts a flow diagram for dataset labeling, in accordance with an embodiment.

FIG. 4A depicts a flow diagram for deployment of the regression model and/or the classification model for performing a library screen or for identifying hits, in accordance with an embodiment.

FIG. 4B depicts a flow diagram for predicting binding affinity using a regression model, in accordance with an embodiment.

FIG. 5A depicts an example structure of a regression model, in accordance with an embodiment.

FIG. 5B depicts an example second model portion of the regression model, in accordance with the embodiment shown in FIG. 5A.

FIG. 5C depicts an example structure of a classification model, in accordance with an embodiment.

FIG. 6A depicts an example flow diagram for training a regression model, in accordance with an embodiment.

FIG. 6B depicts an example flow diagram for training a classification model, in accordance with an embodiment.

FIG. 7A illustrates an example computing device for implementing the system and methods described in FIGS. 1A-1B, 2, 3A-3B, 4A-4B, 5A-5C, and 6A-6B.

FIG. 7B depicts an overall system environment for implementing a compound analysis system, in accordance with an embodiment.

FIG. 7C is an example depiction of a distributed computing system environment for implementing the system environment of FIG. 7B.

FIG. 8 shows an example flow process for conducting a virtual screen, performing hit selection and analysis, or generating binding affinity predictions.

FIG. 9 shows an example diagrammatic representation of a DNA encoded library (DEL) screen. In addition to target specific binding there may be multiple non target binding modes that are sequenced. Additionally, amplification rates are not uniform, leading to noisy count information.

FIG. 10 depicts clustering of datasets following dataset splitting using two different methods.

FIG. 11 depicts an example labeling scheme workflow.

FIG. 12 depicts an example classification model. The classification model uses a GIN-E encoder and maps the encoder output to a single class prediction.

FIG. 13 depicts an example regression model. The regression model has multiple heads, each predicting an enrichment value from a reduced embedding of the encoder output. These enrichments are terms in a sum (with learned weights β) that predict observed counts (UMI).

FIG. 14 depicts performance of classification and regression models for predicting binding affinity.

FIG. 15 depicts performance of regression models for performing a virtual screen.

### DETAILED DESCRIPTION OF THE INVENTION

### Definitions

Terms used in the claims and specification are defined as set forth below unless otherwise specified.

The phrase “obtaining a compound” comprises physically obtaining a compound. “Obtaining a compound” also encompasses obtaining a representation of the compound. Examples of a representation of the compound include a molecular representation such as a molecular fingerprint or a molecular graph. “Obtaining a compound” also encompasses obtaining the compound expressed as a particular structure format. Example structure formats of the compound include any of a simplified molecular-input line-entry system (SMILES) string, MDL Molfile (MDL MOL), Structure Data File (SDF), Protein Data Bank (PDB), Molecule specification file (xyz), International Union of Pure and Applied Chemistry (IUPAC) International Chemical Identifier (InChI), and Tripos Mol2 file (mol2) format.

The phrase “applying the compound as input to a model” comprises implementing a model (e.g., regression model or classification model) to analyze the compound, such as a representation of the compound. In various embodiments, “applying the compound as input to a model” comprises converting a structure format into molecular representations, such as any of a molecular fingerprint or a molecular graph, such that the model analyzes the molecular representation of the compound.

The phrase “selectively expand molecular representations of a training dataset” refers to generating one or more additional molecular representations from a first molecular representation. Generally, the phrase encompasses generating a subset of additional molecular representations from all possible molecular representations. Thus, not all molecular representations are generated for the training dataset. As used herein, selectively expanding molecular representations of a training dataset is referred to as an augmentation. In various embodiments, a tunable hyperparameter controls the implementation of an augmentation, thereby selectively expanding molecular representations of the training dataset such that the model can better handle different compound structure representations, which further improves model performance and generalization.

The phrase “incorporate two or more covariates for predicting the value indicative of binding affinity” generally refers to a machine learning model that is structured to model the effects of two or more covariates. By doing so, the machine learning model predicts a de-noised and de-biased value indicative of binding affinity that is absent the effects of the two or more covariates.

It must be noted that, as used in the specification and the appended claims, the singular forms “a,” “an” and “the” include plural referents unless the context clearly dictates otherwise.

Overview of System Environment

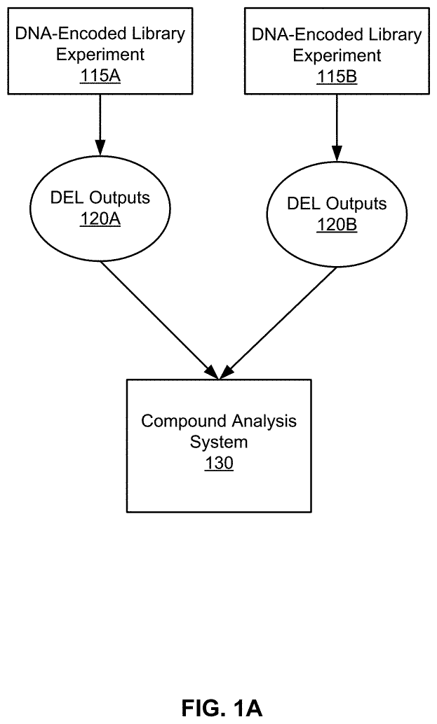

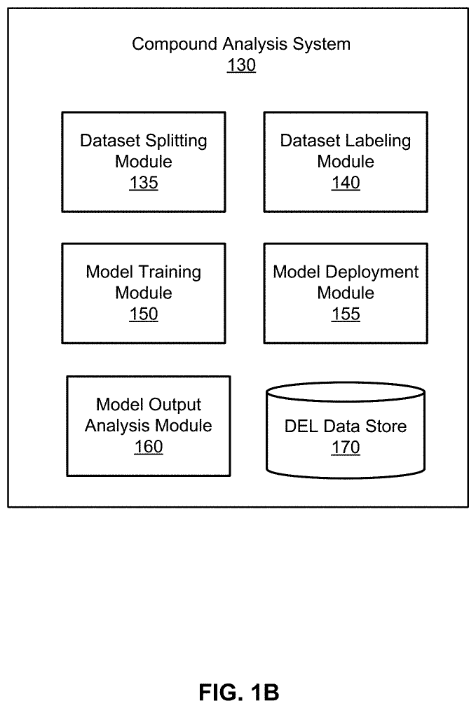

FIG. 1A depicts an example system environment involving a compound analysis system 130, in accordance with an embodiment. In particular, FIG. 1A introduces DNA-encoded library (DEL) experiment 115A and DNA-encoded library (DEL) experiment 115B for generating DEL outputs (e.g., DEL output 120A and DEL output 120B) that are provided to the compound analysis system 130 for training and deploying machine learning models to perform a virtual screen, select and analyze hits, and/or predict binding affinity values. Although FIG. 1A depicts two DEL experiments 115A and 115B, in various embodiments, fewer or additional DEL experiments can be conducted. In various embodiments, the example system environment involves at least three DEL experiments, at least four DEL experiments, at least five DEL experiments, at least six DEL experiments, at least seven DEL experiments, at least eight DEL experiments, at least nine DEL experiments, at least ten DEL experiments, at least fifteen DEL experiments, at least twenty DEL experiments, at least thirty DEL experiments, at least forty DEL experiments, at least fifty DEL experiments, at least sixty DEL experiments, at least seventy DEL experiments, at least eighty DEL experiments, at least ninety DEL experiments, or at least a hundred DEL experiments. The output (e.g., DEL output) of one or more of the DEL experiments can be provided to the compound analysis system for training and deploying machine learning models to perform a virtual screen, select and analyze hits, and/or predict binding affinity values.

In various embodiments, a DEL experiment involves screening small molecule compounds of a DEL library against targets. In various embodiments, a DEL experiment involves pooling small molecule compounds from two or more DEL libraries, and then screening the pooled small molecule compounds from the two or more DEL libraries against targets. In various embodiments, a DEL experiment involves pooling small molecule compounds from three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, or twenty or more DEL libraries, and then screening the pooled small molecule compounds against targets.

In various embodiments, each DEL experiment (e.g., DEL experiments 115A or 115B) can be performed more than once. For example, technical replicates of the DEL experiments can be performed to generate different sets of outputs (e.g., DEL outputs 120A and 120B). For example, DEL experiment 115A can be performed Xtimes, thereby generating XDEL outputs 120A. In various embodiments, the XDEL outputs 120A can be provided to the compound analysis system 130 for their subsequent analysis. For example, the XDEL outputs 120A can be individually analyzed. As another example, the X DEL outputs can be combined into a single DEL output value for subsequent analysis. For example, the X DEL outputs can be averaged into a single DEL output value for subsequent analysis.

Generally, the DEL experiments (e.g., DEL experiments 115A or 115B) involve building small molecule compounds using chemical building blocks, also referred to as synthons. In various embodiments, small molecule compounds can be generated using two chemical building blocks, which are referred to di-synthons. In various embodiments, small molecule compounds can be generated using three chemical building blocks, which are referred to as tri-synthons. In various embodiments, small molecule compounds can be generated using four or more, five or more, six or more, seven or more, eight or more, nine or more, ten or more, fifteen or more, twenty or more, thirty or more, forty or more, or fifty or more chemical building blocks. In various embodiments, a DNA-encoded library (DEL) for a DEL experiment can include at least 10³ unique small molecule compounds. In various embodiments, a DNA-encoded library (DEL) for a DEL experiment can include at least 10⁴ unique small molecule compounds. In various embodiments, a DNA-encoded library (DEL) for a DEL experiment can include at least 10⁵ unique small molecule compounds. In various embodiments, a DNA-encoded library (DEL) for a DEL experiment can include at least 10⁶ unique small molecule compounds. In various embodiments, a DNA-encoded library (DEL) for a DEL experiment can include at least 10⁷ unique small molecule compounds. In various embodiments, a DNA-encoded library (DEL) for a DEL experiment can include at least 10⁸ unique small molecule compounds. In various embodiments, a DNA-encoded library (DEL) for a DEL experiment can include at least 10⁹ unique small molecule compounds. In various embodiments, a DNA-encoded library (DEL) for a DEL experiment can include at least 10¹⁰ unique small molecule compounds. In various embodiments, a DNA-encoded library (DEL) for a DEL experiment can include at least 10¹¹ unique small molecule compounds. In various embodiments, a DNA-encoded library (DEL) for a DEL experiment can include at least 10¹² unique small molecule compounds.

Generally, the small molecule compounds in the DEL are labeled with tags. For example, the small molecule compound can be covalently linked to a unique tag. In various embodiments, the tags include nucleic acid sequences. In various embodiments, the tags include DNA nucleic acid sequences.

In various embodiments, for a DEL experiment (e.g., DEL experiment 115A or 115B), small molecule compounds that are labeled with tags are incubated with immobilized targets. In various embodiments, targets are nucleic acid targets, such as DNA targets or RNA targets. In various embodiments, targets are protein targets. In particular embodiments, protein targets are immobilized on beads. The mixture is washed to remove small molecule compounds that did not bind with the targets. The small molecule compounds that were bound to the targets are eluted and the corresponding tag sequences are amplified. In various embodiments, the tag sequences are amplified through one or more rounds of polymerase chain reaction (PCR) amplification. In various embodiments, the tag sequences are amplified using an isothermal amplification method, such as loop-mediated isothermal amplification (LAMP). The amplified sequences are sequenced to determine a quantitative readout for the number of putative small molecule compounds that were bound to the target. Further details of the methodology of building small molecule compounds of DNA-encoded libraries and methods for identifying putative binders of a DEL target are described in McCloskey, et al. “Machine Learning on DNA-Encoded Libraries: A New Paradigm for Hit Finding.” J. Med. Chem. 2020, 63, 16, 8857-8866, and Lim, K. et al “Machine learning on DNA-encoded library count data using an uncertainty-aware probabilistic loss function.” arXiv: 2108.12471, each of which is hereby incorporated by reference in its entirety.

In various embodiments, for a DEL experiment (e.g., DEL experiment 115A or 115B), small molecule compounds are screened against targets using solid state media that house the targets. Here, in contrast to panning-based systems which used immobilized targets on beads, targets are incorporated into the solid state media. For example, this screen can involve running small molecule compounds of the DEL through a solid state medium such as a gel that incorporates the target using electrophoresis. The gel is then sliced to obtain tags that were used to label small molecule compounds. The presence of a tag suggests that the small molecule compound is a putative binder to the target that was incorporated in the gel. The tags are amplified (e.g., through PCR or an isothermal amplification process such as LAMP) and then sequenced. Further details for gel electrophoresis methodology for identifying putative binders is described in International Patent Application No. PCT/US2020/022662, which is hereby incorporated by reference in its entirety.

In various embodiments, one or more of the DNA-encoded library experiments 115 are performed to model one or more covariates. Generally, a covariate refers to an experimental influence that impacts a DEL readout (e.g., a DEL output) of a DEL experiment, and therefore serves as a confounding factor in determining the actual binding between a small molecule compound and a target. Example covariates include, without limitation, non-target specific binding (e.g., binding to beads, binding to streptavidin of the beads, binding to biotin, binding to gels, binding to DEL container surfaces, binding to tags e.g., DNA tags or protein tags), enrichment in other negative control pans, enrichment in other target pans as indication for promiscuity, compound synthesis yield, reaction type, starting tag imbalance, initial load populations, experimental conditions, chemical reaction yields, side and truncated products, errors from the library synthesis, DNA affinity to target, sequencing depth, and sequencing noise such as PCR bias.

To provide an example, a DEL experiment 115 may be designed to model the covariate of small molecule compound binding to beads. Here, if a small molecule compound binds to a bead instead of or in addition to the immobilized target on the bead, the subsequent washing and eluting step may result in the detection and identification of the small molecule compound as a putative binder, even though the small molecule compound does not bind specifically to the target. Thus, a DEL experiment 115 for modeling the covariate of non-specific binding to beads may involve incubating small molecule compounds with beads without the presence of immobilized targets on the bead. The mixture of the small molecule compound and the beads is washed to remove non-binding compounds that did not bind with the beads. The small molecule compounds bound to beads are eluted and the corresponding tag sequences are amplified (e.g., amplified through PCR or isothermal amplification such as LAMP). The amplified sequences are sequenced to determine a quantitative readout for the number of small molecule compounds that were bound to the bead. Thus, this quantitative readout can be a DEL output (e.g., DEL output 120) from a DEL experiment (e.g., DEL experiment 115) that is then provided to the compound analysis system 130.

As another example, a DEL experiment 115 may be designed to model the covariate of small molecule compound binding to streptavidin linkers on beads. Here, the streptavidin linker on a bead is used to attach the target (e.g., target protein) to a bead. If a small molecule compound binds to the streptavidin linker instead of or in addition to the immobilized target on the bead, the subsequent washing and eluting step may result in the detection and identification of the small molecule compound as a putative binder, even though the small molecule compound does not bind specifically to the target. Thus, a DEL experiment 115 for modeling the covariate of non-specific binding to beads may involve incubating small molecule compounds with streptavidin linkers on beads without the presence of immobilized targets on the bead. The mixture of the small molecule compound and the streptavidin linker on beads is washed to remove non-binding compounds. The small molecule compounds bound to streptavidin linker on beads are eluted and the corresponding tag sequences are amplified (e.g., amplified through PCR or isothermal amplification such as LAMP). The amplified sequences are sequenced to determine a quantitative readout for the number of small molecule compounds that were bound to the streptavidin linkers on beads. Thus, this quantitative readout can be a DEL output (e.g., DEL output 120) from a DEL experiment (e.g., DEL experiment 115) that is then provided to the compound analysis system 130.

As another example, a DEL experiment 115 may be designed to model the covariate of small molecule compound binding to a gel, which arises when implementing the nDexer methodology. Here, if a small molecule compound binds to the gel during electrophoresis instead of or in addition to the immobilized target on the bead, the subsequent washing and eluting step may result in the detection and identification of the small molecule compound as a putative binder, even though the small molecule compound does not bind to the target. Thus, the DEL experiment 115 may involve incubating small molecule compounds with control gels that do not incorporate the target. The small molecule compounds bound or immobilized within the gel are eluted and the corresponding tag sequences are amplified (e.g., amplified through PCR or isothermal amplification such as LAMP). The amplified sequences are sequenced to determine a quantitative readout for the number of small molecule compounds that were bound or immobilized in the gel. Thus, this quantitative readout can be a DEL output (e.g., DEL output 120) from a DEL experiment (e.g., DEL experiment 115) that is then provided to the compound analysis system 130.

In various embodiments, at least two of the DEL experiments 115 are performed to model at least two covariates. In various embodiments, at least three DEL experiments 115 are performed to model at least three covariates. In various embodiments, at least four DEL experiments 115 are performed to model at least four covariates. In various embodiments, at least five DEL experiments 115 are performed to model at least five covariates. In various embodiments, at least six DEL experiments 115 are performed to model at least six covariates. In various embodiments, at least seven DEL experiments 115 are performed to model at least seven covariates. In various embodiments, at least eight DEL experiments 115 are performed to model at least eight covariates. In various embodiments, at least nine DEL experiments 115 are performed to model at least nine covariates. In various embodiments, at least ten DEL experiments 115 are performed to model at least ten covariates. The DEL outputs from each of the DEL experiments can be provided to the compound analysis system 130. In various embodiments, the DEL experiments 115 for modeling covariates can be performed more than once. For example, technical replicates of the DEL experiments 115 for modeling covariates can be performed. In particular embodiments, at least three replicates of the DEL experiments 115 for modeling covariates can be performed.

The DEL outputs (e.g., DEL output 120A and/or DEL output 120B) from each of the DEL experiments can include DEL readouts for the small molecule compounds of the DEL experiment. In various embodiments, a DEL output can be a DEL count for the small molecule compounds of the DEL experiment. Thus, small molecule compounds that are putative binders of a target would have higher DEL counts in comparison to small molecule compounds that are not putative binders of the target. As an example, a DEL count can be a unique molecular index (UMI) count determined through sequencing. As an example, a DEL count may be the number of counts observed in a particular index of a solid state media (e.g., a gel). In various embodiments, a DEL output can be DEL reads corresponding to the small molecule compounds. For example, a DEL read can be a sequence read derived from the tag that labeled a corresponding small molecule compound. In various embodiments, a DEL output can be a DEL index. For example, a DEL index can refer to a slice number of a solid state media (e.g., a gel) which indicates how far a DEL member traveled down the solid state media.

Generally, the compound analysis system 130, trains and/or deploys machine learning models to perform a virtual screen, select and analyze hits, and/or predict binding affinity values. In various embodiments, the machine learning models include one or more regression models. In various embodiments, the machine learning models include one or more classification models. In various embodiments, the machine learning models include one or more regression models and one or more classification models. The compound analysis system 130 trains machine learning models using at least the DEL outputs (e.g., DEL outputs 120A and 120B) that are derived from the DEL experiments (e.g., DEL experiments 115A and 115B).

As further described herein, the compound analysis system 130 can train a classification model and/or a regression model, each of which can be deployed for performing a virtual screen, selecting and analyzing hits, and/or predicting binding affinity values. In particular embodiments, the compound analysis system 130 trains the classification model and/or a regression model using an augmentation technique that selectively expands molecular representations of a training dataset used to train the classification model and/or the regression model. For example, the classification model and/or the regression model may include a tunable hyperparameter representing a probability that controls augmentation of compound structure representations to selectively expand molecular representations of the training dataset. Altogether, the tunable hyperparameter controls implementation of the augmentations, thereby selectively expanding molecular representations of the training dataset such that the model can better handle different compound structure representations, which further improves model performance and generalization.

In particular embodiments, the compound analysis system 130 trains the regression model to incorporate one or more covariates for predicting a value indicative of binding affinity between compounds and targets. In particular embodiments, the compound analysis system 130 trains the regression model to incorporate two or more covariates for predicting a value indicative of binding affinity between compounds and targets. Put more generally, the compound analysis system 130 trains a regression model such that the regression model is able to better predict de-noised and de-biased values (e.g., enrichment predictions) that are indicative of binding affinity between a compound and target.

FIG. 1B depicts a block diagram of the compound analysis system 130, in accordance with an embodiment. The compound analysis system 130 in FIG. 1B is shown to introduce a dataset splitting module 135, dataset labeling module 140, model training module 150, model deployment module 155, model output analysis module 160, and a DEL data store 170.

Referring to the dataset splitting module 135, it performs splitting of a dataset. In various embodiments, the dataset splitting module 135 splits the dataset into a training dataset and a validation dataset. In various embodiments, the dataset splitting module 135 splits the dataset into a training dataset, a validation dataset, and a test dataset. Therefore, the training dataset can be used to train machine learning models (e.g., classification model or regression model), the validation dataset can be used to validate machine learning models, and the test dataset can be used to test the performance of machine learning models. In various embodiments, the dataset can be split into one or more validation datasets. For example, the dataset can be split into k different validation datasets. Therefore, the k different validation datasets can be used to perform k-folds cross validation.

In various embodiments, the dataset can be a DEL dataset comprising DEL outputs derived from multiple DEL experiments. The DEL dataset may be stored and retrieved from the DEL data store 170. In various embodiments, the DEL outputs from multiple DEL experiments can be pooled, thereby enlarging the total number of small molecule compounds that have been experimentally modeled. The dataset splitting module 135 selectively splits the pooled DEL outputs to generate a training dataset for training machine learning models and a validation dataset for validating machine learning models. In various embodiments, the dataset splitting module 135 may split a dataset into a training dataset and a validation dataset based on the DEL experiments that the dataset was obtained from. For example, the dataset splitting module 135 may divide a training dataset and a validation dataset such that the training dataset is derived from a first DEL experiment and the validation dataset is derived from a second DEL experiment. Therefore, the machine learning model is trained and validated on different datasets that derive from different DEL experiments which may prevent overfitting of the model. Further details of the methods performed by the dataset splitting module 135 are described herein.

Referring to the dataset labeling module 140, it labels the training and validation datasets using a plurality of labels and selects the top-performing labels. In various embodiments, the dataset labeling module 140 selects top-performing labels by evaluating performance of trained label prediction models. Here, different label prediction models may be trained using different subsets of the plurality of labels. Then, the label prediction models are evaluated according to performance metrics (e.g., Boltzmann-Enhanced Discrimination of Receiver Operating Curve (BEDROC) metric, an Area Under ROC (AUROC) metric, or an average precision (AVG-PRC) metric). Once the dataset labeling module 140 selects the top-performing labels, machine learning models (e.g., a regression model and/or classification model) can be trained using the top-performing labels (e.g., through supervised training). Further details of the methods performed by the dataset labeling module 140 are described herein.

Referring to the model training module 150, it trains machine learning models using a training dataset. For example, the model training module 150 trains a regression model using a training dataset. In various embodiments, the training dataset is unlabeled. In various embodiments, the training dataset is labeled. In particular embodiments, the training dataset is labeled using DEL counts (e.g., UMI counts). In such embodiments, the model training module 150 trains a regression model using the labeled training dataset through supervised learning. In particular embodiments, the labeled training dataset used to train the regression model need not undergo the labeling process described herein with respect to the dataset labeling module 140.

As another example, the model training module 150 trains a classification model using a training dataset. In various embodiments, the training dataset is labeled using the top-performing labels identified by the dataset labeling module 140. The model training module 150 may further validate the trained machine learning models using validation datasets, such as a labeled or unlabeled validation dataset. Further details of the training processes performed by the model training module 150 are described herein.

Referring to the model deployment module 155, it deploys machine learning models such as one or more regression models and/or one or more classification models, to perform a virtual screen, select and analyze hits, and/or predict binding affinity values between compounds and targets. In particular embodiments, the model deployment module 155 deploys both a regression model and a classification model to perform a virtual screen or to select and analyze hits. In particular embodiments, the model deployment module 155 deploys a regression model to predict binding affinity values between compounds and targets. Further details of the processes performed by the model deployment module 155 are described herein.

Referring to the model output analysis module 160, it analyzes the outputs of one or more machine learned models. In various embodiments, the model output analysis module 160 translates predictions outputted by one or more machine learned models to binding affinity values. As a specific example, the model output analysis module 160 may translate an enrichment prediction outputted by the regression model to a binding affinity value. In various embodiments, the model output analysis module 160 identifies candidate compounds that are likely binders of a target based on the outputs of one or more machine learned models. For example, the model output analysis module 160 identifies candidate compounds likely to bind to a target that represent overlapping compounds predicted to be binders by the classification model and by the regression model. Thus, one or more of the candidate compounds can be synthesized e.g., as part of a medicinal chemistry campaign. The one or more candidate compounds can be synthesized and experimentally screened against the target to validate its binding and effects. Further details of the processes performed by the model output analysis module 160 are described herein.

### DEL Dataset Splitting and/or Labeling

Embodiments disclosed herein involve generating training datasets and validation datasets for training and evaluating machine learning models. In various embodiments, embodiments disclosed herein further involve generating test datasets for testing machine learning models. In particular embodiments, training datasets and validation datasets are generated from a DEL dataset that is derived from one or more DEL experiments. For example, the training datasets and validation datasets can be generated from a DEL dataset comprising DEL outputs (e.g., DEL outputs 120A or 120B) from multiple DEL experiments (e.g., DEL experiment 115A or 115B). In various embodiments, DEL datasets are split to generate the training dataset and validation dataset. In various embodiments, training datasets and validation datasets are further labeled using top-performing labels that are selected by evaluating performance of trained label prediction models. Generally, the steps described herein for splitting the DEL dataset into a training dataset and validation dataset are performed by the dataset splitting module 135 (see FIG. 1B). Additionally, the steps described herein for labeling the training dataset and validation dataset and selecting top-performing labels are performed by the dataset labeling module 140 (see FIG. 1B). In various embodiments, the training dataset and validation dataset are used to train and validate, respectively, a regression model. In various embodiments, the labeled training dataset and labeled validation dataset are used to train and validate, respectively, a classification model. In various embodiments, a test dataset can be used to evaluate performance of a regression model or a classification model.

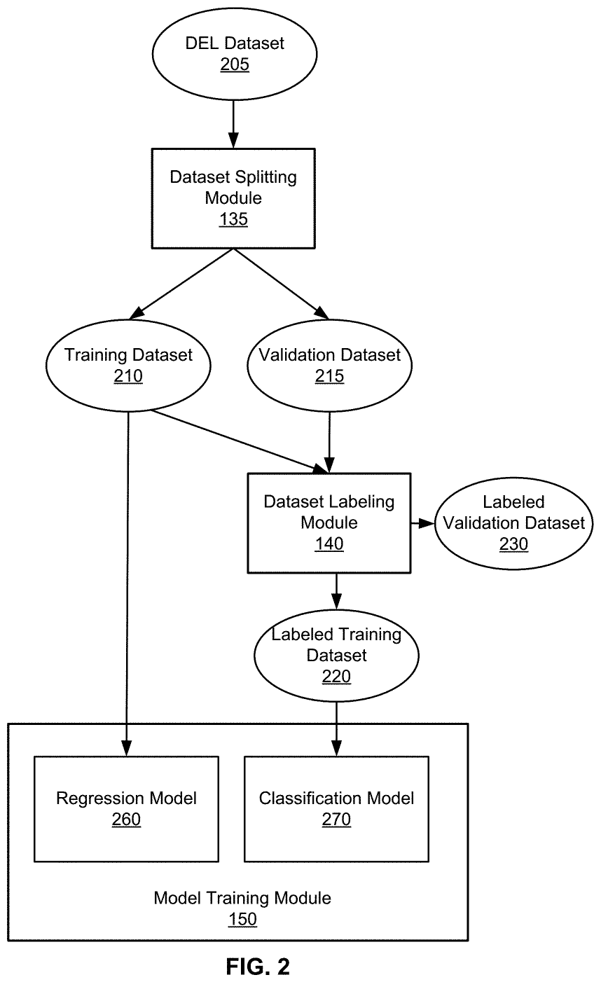

FIG. 2 depicts a flow diagram for splitting and/or labeling a DNA encoded library (DEL) dataset for training a regression model or a classification model, in accordance with an embodiment. Specifically, FIG. 2 is shown to introduce the flow process for generating a training dataset 210, validation dataset 215, labeled training dataset 220, and labeled validation dataset 230 for use in training and validating a regression model 260 and classification model 270. Although not shown, embodiments may further include generating a test dataset and/or a labeled test dataset for use in testing a regression model 260 and classification model 270.

The flow diagram in FIG. 2 begins with a DEL dataset 205. Here, the DEL dataset 205 can include DEL outputs (e.g., DEL outputs 120A and/or 120B) from one or more DEL experiments (e.g., DEL experiment 115A and/or 115B). For example, the DEL outputs from multiple DEL experiments can be pooled such that the DEL dataset 205 includes a larger number of DEL outputs corresponding to experimentally tested small molecule compounds. In various embodiments, the DEL dataset 205 further includes identities of the small molecule compounds corresponding to the DEL outputs. For example, the DEL dataset 205 can include molecular representations (e.g., molecular fingerprint or molecular graph) of the small molecule compounds that were previously tested in the DEL experiments. In various embodiments, the DEL dataset 205 includes identities of building blocks (e.g., synthons, disynthons, or trisynthons) or the small molecule compounds corresponding to the DEL outputs. The identities of the building blocks or identities of the small molecule compounds may be arranged in the DEL dataset 205 to correspond with their respective DEL outputs. For example, identities of building blocks or identities of compounds may be arranged in a first column and the corresponding DEL output (e.g., DEL counts, DEL reads, or DEL indices) for each compound may be arranged in a second column that is adjacent to the first column. Each small molecule compound and DEL output pairing can be referred to as an example, such as a training example or validation example. In various embodiments, an example, such as a training example or validation example, can include further information beyond the pairing of the small molecule compound and corresponding DEL output.

The DEL dataset 205 is analyzed by the dataset splitting module 135 which generates a training dataset 210 and a validation dataset 215. In various embodiments, the dataset splitting module 135 may divide the DEL dataset 205 into the training dataset 210 and the validation dataset 215 based on the DEL experiments that generated the DEL outputs. For example, the dataset splitting module 135 may divide DEL outputs from a first set of DEL experiments into the training dataset 210 and may divide DEL outputs from a second set of DEL experiments into the validation dataset 215. Thus, a machine learning model is trained based on DEL experimental data that is independent from DEL experimental data that is used to validate the machine learning model.

In particular embodiments, small molecule compounds of the DEL dataset 205 are selectively split into the training dataset 210 and the validation dataset 215 such that at least a threshold percentage of structures of compounds that are present in the training dataset 210 are different from the structure of compounds that are present in the validation dataset 215. As one example, a structure of a compound refers to a building block (e.g., a synthon) of a compound. In particular embodiments, small molecule compounds of the DEL dataset 205 are selectively split into the training dataset 210 and the validation dataset 215 such that at least a threshold percentage of compounds that are present in the training dataset 210 are different from the compounds that are present in the validation dataset 215. Generally, selectively splitting small molecule compounds into the training dataset 210 and validation dataset 215 enables the evaluation of the machine learning model's ability to generalize to new chemical domains. In other words, a machine learning model is trained on a training dataset 210 including structures of compounds and is further validated for its ability to accurately generate predictions based on previously unseen structures of compounds of the validation dataset 215. Standard methods like random splitting, Bemis-Murcko splitting, and Taylor-Butina clustering cannot achieve the selective splitting of compounds in the training dataset 210 and validation dataset 215 described herein likely due to the combinatorial nature of the DEL or due to the inability to scale to the hundreds of millions of compounds typically in the DEL.

In various embodiments, at least 10% of the building blocks of compounds present in the training dataset 210 are different from the building blocks of compounds that are present in the validation dataset 215. In various embodiments, at least 20% of the building blocks of compounds present in the training dataset 210 are different from the building blocks of compounds that are present in the validation dataset 215. In various embodiments, at least 30% of the building blocks of compounds present in the training dataset 210 are different from the building blocks of compounds that are present in the validation dataset 215. In various embodiments, at least 40% of the building blocks of compounds present in the training dataset 210 are different from the building blocks of compounds that are present in the validation dataset 215. In various embodiments, at least 50% of the building blocks of compounds present in the training dataset 210 are different from the building blocks of compounds that are present in the validation dataset 215. In various embodiments, at least 60% of the building blocks of compounds present in the training dataset 210 are different from the building blocks of compounds that are present in the validation dataset 215. In various embodiments, at least 70% of the building blocks of compounds present in the training dataset 210 are different from the building blocks of compounds that are present in the validation dataset 215. In various embodiments, at least 80% of the building blocks of compounds present in the training dataset 210 are different from the building blocks of compounds that are present in the validation dataset 215. In various embodiments, at least 90% of the building blocks of compounds present in the training dataset 210 are different from the building blocks of compounds that are present in the validation dataset 215. In various embodiments, at least 95% of the building blocks of compounds present in the training dataset 210 are different from the building blocks of compounds that are present in the validation dataset 215.

In various embodiments, compounds are determined to be different from one another by comparing the molecular fingerprints of the compounds. In particular embodiments, a first compound is different from a second compound if the distance between the molecular fingerprint of the first compound and the molecular fingerprint of the second compound is greater than a threshold distance. For example, a distance between molecular fingerprints can be measured according to Tanimoto distance. In various embodiments, the threshold distance is a distance of X. In various embodiments, X can be a value of 0.1, 0.2, 0.3, 0.4, 0.5, 0.6, 0.7, 0.8, 0.9, or 1.0. In particular embodiments, X is a value of 0.7. In various embodiments, at least 10% of the building blocks of compounds present in the training dataset 210 are not present in the validation dataset 215. In various embodiments, at least 10%, at least 20%, at least 30%, at least 40%, at least 50%, at least 60%, at least 70%, at least 80%, at least 90%, or at least 95% of the building blocks of compounds present in the training dataset 210 are not present in the validation dataset 215.

To split the DEL dataset 205 into the training dataset 210 and validation dataset 215, the dataset splitting module 135 generates a representative sample of compounds from the DEL. The dataset splitting module 135 ensures that at least a threshold number of building blocks of the DEL synthesis are present in the representative sample. In various embodiments, the threshold number of building blocks is 1 building block. In various embodiments, the threshold number of building blocks is 10¹ building blocks. In various embodiments, the threshold number of building blocks is 10² building blocks. In various embodiments, the threshold number of building blocks is 10³ building blocks. In various embodiments, the threshold number of building blocks is 10⁴ building blocks. In various embodiments, the threshold number of building blocks is 10⁵ building blocks. In various embodiments, the threshold number of building blocks is 10⁶ building blocks. In various embodiments, the threshold number of building blocks is 10⁷ building blocks. In various embodiments, the threshold number of building blocks is 10⁸ building blocks. In various embodiments, the threshold number of building blocks is 10⁹ building blocks. In various embodiments, the threshold number of building blocks is 10¹⁰ building blocks. In various embodiments, the threshold number of building blocks is 50% of the total number of building blocks used in the DEL synthesis. In various embodiments, the threshold number of building blocks is 60% of the total number of building blocks used in the DEL synthesis. In various embodiments, the threshold number of building blocks is 70% of the total number of building blocks used in the DEL synthesis. In various embodiments, the threshold number of building blocks is 80% of the total number of building blocks used in the DEL synthesis. In various embodiments, the threshold number of building blocks is 90% of the total number of building blocks used in the DEL synthesis. In various embodiments, the threshold number of building blocks is 91%, 92%, 93%, 94%, 95%, 96%, 97%, 98%, 99%, or 100% of the total number of building blocks used in the DEL synthesis. In particular embodiments, the threshold number of building blocks is 95% of the total number of building blocks used in the DEL synthesis. In particular embodiments, the threshold number of building blocks is 100% of the total number of building blocks used in the DEL synthesis.

Once the representative sample is generated, the dataset splitting module 135 performs clustering on the compounds in the representative sample. In various embodiments, the dataset splitting module 135 performs hierarchical clustering on molecular representations of the compounds in the representative sample. Examples of hierarchical clustering include DBScan, HDBScan, ward clustering, and single linkage clustering. In various embodiments, the dataset splitting module 135 performs non-hierarchical clustering on molecular representations of the compounds in the representative sample. Example of non-hierarchical clustering include Sphere exclusion, Butina clustering, and k-means clustering. In various embodiments, the compounds in the representative sample are clustered into two or more groups. In various embodiments, the compounds in the representative sample are clustered into five or more, ten or more, fifteen or more, twenty or more, twenty five or more, thirty or more, forty or more, fifty or more, sixty or more, seventy or more, eighty or more, ninety or more, or a hundred or more groups. In particular embodiments, the compounds in the representative sample are clustered into 100 groups.

The dataset splitting module 135 incorporates the additional DEL compounds that were not included in the representative sample. For example, the dataset splitting module 135 incorporates the additional DEL compounds into one of the two or more groups. In various embodiments, for an additional DEL compound, the dataset splitting module 135 queries the representative sample to identify a corresponding DEL compound representing the nearest neighbor of the additional DEL compound.

In various embodiments, neighboring compounds are identified by representing the compounds as a molecular representation, an example of which includes a Morgan fingerprint. A similarity or distance metric is then calculated between the two compounds. For example, a similarity metric can be Tanimoto Similarity and a distance metric can be Jaccard distance, both of which measure the similarity between the molecular fingerprints of the two compounds. A nearest neighbor of a first compound is a second compound with the highest similarity or the lowest distance to the first compound. Thus, the dataset splitting module 135 incorporates the additional DEL compound into the group of the nearest neighbor DEL compound. In various embodiments, as a result of this procedure, the dataset splitting module 135 has assigned every additional DEL compound to one of the two or more groups. In various embodiments, as a result of this procedure, the dataset splitting module 135 has assigned at least 10² additional DEL compounds to one of the two or more groups. In various embodiments, as a result of this procedure, the dataset splitting module 135 has assigned at least 10³ additional DEL compounds to one of the two or more groups. In various embodiments, as a result of this procedure, the dataset splitting module 135 has assigned at least 10⁴ additional DEL compounds to one of the two or more groups. In various embodiments, as a result of this procedure, the dataset splitting module 135 has assigned at least 10⁵ additional DEL compounds to one of the two or more groups. In various embodiments, as a result of this procedure, the dataset splitting module 135 has assigned at least 10⁶ additional DEL compounds to one of the two or more groups. In various embodiments, as a result of this procedure, the dataset splitting module 135 has assigned at least 10⁷ additional DEL compounds to one of the two or more groups. In various embodiments, as a result of this procedure, the dataset splitting module 135 has assigned at least 10⁹ additional DEL compounds to one of the two or more groups. In various embodiments, as a result of this procedure, the dataset splitting module 135 has assigned at least 10¹⁰ additional DEL compounds to one of the two or more groups.

The dataset splitting module 135 generates the training dataset 210 and validation dataset 215 from the two or more groups. In various embodiments, the dataset splitting module 135 assigns groups to either the training dataset 210 or the validation dataset 215 to achieve a desired split. In one embodiment, the dataset splitting module 135 assigns groups such that about 60% of the original dataset (e.g., DEL dataset 205) is the training dataset 210 and the about remaining 40% of the original dataset (e.g., DEL dataset 205) is the validation dataset 215. In various embodiment, the dataset splitting module 135 assigns groups such that about 70% of the original dataset (e.g., DEL dataset 205) is the training dataset 210 and the about remaining 30% of the original dataset (e.g., DEL dataset 205) is the validation dataset 215. In one embodiment, the dataset splitting module 135 assigns groups such that about 80% of the original dataset (e.g., DEL dataset 205) is the training dataset 210 and the about remaining 20% of the original dataset (e.g., DEL dataset 205) is the validation dataset 215.

In various embodiments, the dataset splitting module 135 assigns groups to either the training dataset 210 or the validation dataset 215 based on labels of the original dataset (e.g., DEL dataset 205). For example, labels of the DEL dataset 205 may be binary labels that identify binders and non-binders. As another example, labels of the DEL dataset 205 may be multi-class labels. Multi-class labels can differentiate types of binders or types of non-binders. For example, multi-class labels can include strong binder, weak binder, non-binder, or off target binder. In such embodiments, the dataset splitting module 135 assigns groups to either the training dataset 210 or the validation dataset 215 based on the labels to ensure that balanced label proportions are present in the training dataset 210 and the validation dataset 215. For example, dataset splitting module 135 assigns groups to either the training dataset 210 or the validation dataset 215 such that the training dataset 210 and/or the validation dataset 215 include a 50:50 split of binders and non-binders. For example, dataset splitting module 135 assigns groups to either the training dataset 210 or the validation dataset 215 such that the training dataset 210 and/or the validation dataset 215 include a 30:70 split, a 40:60 split, a 60:40 split, or a 70:30 split of binders and non-binders.

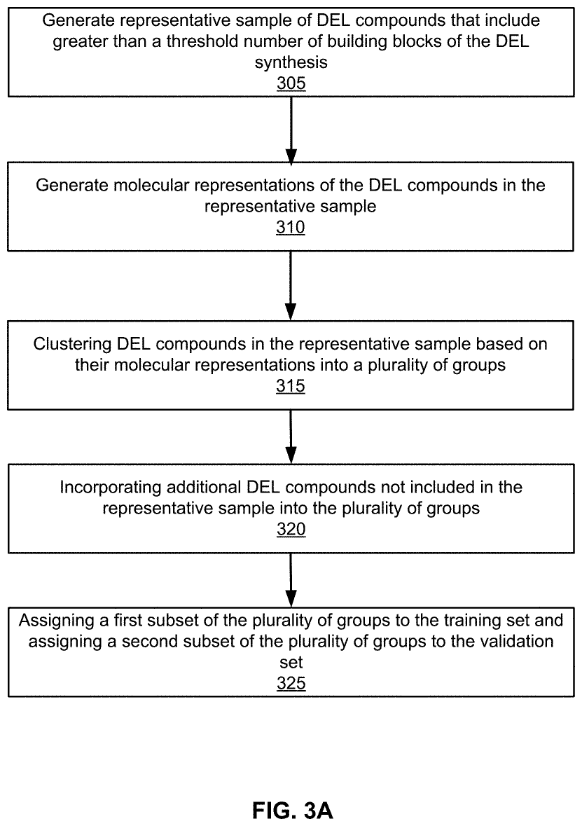

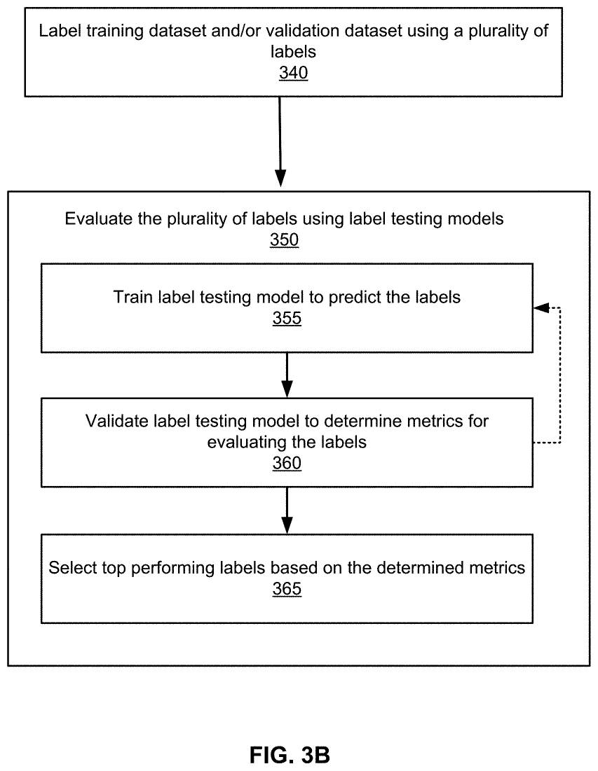

FIG. 3A depicts a flow process for dataset splitting, in accordance with an embodiment. Step 305 involves generating a representative sample of DEL compounds that include greater than a threshold number of building blocks of the DEL synthesis. Step 310 involves generating molecular representations of the DEL compounds in the representative sample. Step 315 involves clustering the DEL compounds in the representative sample based on their molecular representations into a plurality of groups. In various embodiments, step 315 involves performing hierarchical clustering on the molecular representations of the DEL compounds to generate groups of the DEL compounds. Step 320 involves further incorporating additional DEL compounds that were not included in the representative sample. Specifically, the additional DEL compounds are incorporated into the different groups based on the additional DEL compound's nearest neighbor that is present in the representative sample. In various embodiments, after this step 320, all DEL compounds in the DEL are assigned to one group. Step 325 involves assigning a first subset of the groups to the training set and a second subset of the groups to the validation set. Therefore, the training set can be used to train the machine learning models (e.g., regression model and/or classification model) and the validation can be used to validate/evaluate the machine learning models (e.g., regression model and/or classification model). In various embodiments, the training set and validation set can further undergo dataset labeling, as is described in further detail herein.

Referring again to FIG. 2, the training dataset 210 can be used to train the regression model 260. The validation dataset 215 can then be used to validate the regression model 260. As shown in FIG. 2, the training dataset 210 may be directly used to train the regression model 260. In various embodiments, the training dataset 210 can be further processed prior to being used to train the regression model. For example, the training dataset 210 can be processed which can then be used to train the regression model 260. In various embodiments, processing the training dataset 210 involves obtaining denoised counts/reads/indices. As a specific example, the training dataset 210 can be processed using a generalized linear mixed model (GLMM), such as a linear mixed model or a poisson mixed model. Here, the GLMM models the random effects associated with synthon enrichment, thereby denoising counts/reads/indices. In various embodiments, the GLMM models the random effects associated with synthon enrichment due to target binding as opposed to non-target binding. This can be useful for particular applications, such as for hit picking within a library. Thus, the denoised counts/reads/indices from the linear mixed model can be provided to the regression model 260. In particular embodiments, the denoised counts/reads/indices serve as ground truth labels for training the regression model 260. Further details for training and validating the regression model 260 are described herein.

In various embodiments, the training dataset 210 and the validation dataset 215 may be labeled by the dataset labeling module 140 to generate a labeled training dataset 220 and a labeled validation dataset 230. Thus, the labeled training dataset 220 can be used to train the classification model 270, which is further validated using the labeled validation dataset 230.

In various embodiments, the dataset labeling module 140 labels the training dataset 210 and validation dataset 215 with various labels (e.g., fixed or preassigned labels) and selects the top-performing labels. Thus, the labeled training dataset and labeled validation dataset corresponding to the top-performing labels can be subsequently used to train a machine learning model (e.g., classification model).

In various embodiments, the dataset labeling module 140 identifies the top performing labels by differently labeling datasets, and then training/evaluating label testing machine learning models using the datasets to determine the performance of the different labels. In various embodiments, the dataset labeling module 140 differently labels the training dataset 210 and validation dataset 215 to identify the top performing labels. In various embodiments, the dataset labeling module 140 differently labels datasets other than the training dataset 210 and the validation dataset 215 to identify the top performing labels.

Although the description below is in reference to the dataset labeling module 140, in various embodiments, one or more other modules can be deployed to identify top performing labels by differently labeling datasets and training/evaluating label testing machine learning models to determine the performance of the different labels. For example, the one or more other modules can represent submodules of the dataset labeling module 140. As another example, the one or more other modules can represent separate modules distinct from the dataset labeling module 140. Thus, the steps of labeling the training dataset 210 and validation dataset 215 (performed by the dataset labeling module 140) and the steps of identifying top performing labels can be performed by different modules.

In various embodiments, the dataset labeling module 140 trains the label testing machine learning models using the differently labeled versions of the training dataset 210. In various embodiments, the dataset labeling module 140 evaluates the label testing machine learning models using the differently labeled versions of the validation dataset 215. In various embodiments, the dataset labeling module 140 evaluates the label testing machine learning models using subsets of the differently labeled versions of the validation dataset 215. In various embodiments, the dataset labeling module 140 evaluates the label testing machine learning models using a labeled dataset different from the validation dataset 215. For example, the validation dataset 215 is used to evaluate the regression model 260 and/or classification model 270 and therefore, a different labeled dataset is used to evaluate the label testing machine learning models.

For a classification task, the dataset labeling module 140 differently labels the training dataset 210 and/or validation dataset 215 using labels based on various thresholds. In various embodiments, a single threshold can be implemented for a binary classification. For example, for a given threshold, the dataset labeling module 140 labels an example in the training dataset 210 as a member of the first class if the value is above the threshold. Alternatively, the dataset labeling module 140 labels an example in the training dataset 210 as a member of the second class if the value is below the threshold. In various embodiments, additional thresholds can be implemented for a multi-classification. For example, two thresholds can be implemented for distinguishing three classes.

In various embodiments, the N different thresholds can be established using classical statistics that incorporate one or more covariates. For example, a threshold can be developed according to enrichment scores over covariates such as starting tag imbalance and off target signal. In various embodiments, the threshold can be the difference between a target enrichment score and the sum of the starting tag imbalance and off target signal. In various embodiments, the N different thresholds can be established using a generalized linear mixed model, which incorporates learning of the various covariates. In various embodiments, the different thresholds can be implemented in successive steps. For example, two thresholds can be implemented through a two step thresholding process. Therefore, a label can be assigned if the value passes both a first threshold and a second threshold.

In various embodiments, the dataset labeling module 140 labels the training dataset 210 the validation dataset 210 using at least N different thresholds. Therefore, for each training example in the training dataset 210, the dataset labeling module 140 generates N different labels according to the N different thresholds. In various embodiments, N is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 60, at least 70, at least 80, at least 90, at least 100, at least 150, at least 200, at least 250, at least 500, or at least 1000.

Examples of threshold values can include any of 2 counts, 3 counts, 4 counts, 5 counts, 6 counts, 7 counts, 8 counts, 9 counts, 10 counts, 15 counts, 20 counts, 30 counts, 40 counts, or 50 counts. To provide a specific example, assume N=3 different threshold values of 10, 30, and 50 counts. Assume the training example in the training dataset identifies that a small molecule compounds corresponds to a value of 40 counts (e.g., DEL counts). The dataset labeling module 140 compares the value of the example (e.g., 40 counts) to each of the 3 different thresholds and labels the training example with 3 corresponding labels. For example, given that 40 counts is greater than the first threshold of 10 counts, the dataset labeling module 140 assigns a first label of “1” indicating membership in a first class. Additionally, given that 40 counts is greater than the second threshold of 30 counts, the dataset labeling module 140 assigns a second label of “1” indicating membership in a first class. Additionally, given that 40 counts is less than the third threshold of 50 counts, the dataset labeling module 140 assigns a third label of “0” indicating membership in a second class. The dataset labeling module 140 can repeat this process of labeling for the training examples in the training dataset 210 and the validation dataset 215.

In various embodiments, a two-step process is implemented to generate a label. A first step involves comparing the counts to a first threshold. If the counts are below the first threshold, then the dataset labeling module 140 does not assign a label. If the counts are above the first threshold, then the dataset labeling module 140 assigns a label according to a second threshold. For example, if the count is above the first threshold and also the second threshold, then the dataset labeling module 140 assigns a label indicative of membership in a first class. If the count is above the first threshold and below the second threshold, then the dataset labeling module 140 assigns a label indicative of membership in a second class.

The dataset labeling module 140 evaluates the N different labels for the training examples of the training dataset 210 using label prediction models. In various embodiments, label prediction models are machine learning models. Examples of machine learning models can include any of a regression model (e.g., linear regression, logistic regression, or polynomial regression), decision tree, random forest, support vector machine, Naïve Bayes model, k-means cluster, or neural network (e.g., feed-forward networks, convolutional neural networks (CNN), deep neural networks (DNN), autoencoder neural networks, generative adversarial networks, or recurrent networks (e.g., long short-term memory networks (LSTM), bi-directional recurrent networks, deep bi-directional recurrent networks, and transformer models). In particular embodiments, the label prediction models are random forest models. Generally, the label prediction models are trained using assigned labels of the training dataset 210. For example, assuming N different labels, N different testing models are separately trained using the N different labels of the training dataset. The label prediction models can then be evaluated using labeled validation data (e.g., either a subset of a labeled version of the validation dataset 215, or an entirely different labeled validation dataset e.g., a different validation dataset with fixed labels).

In various embodiments, the training of the label prediction model involves providing a labeled training example of the training dataset. In various embodiments, the labeled training example can include the small molecule compound that is expressed in a particular structure format. For example, the small molecule compound can be represented as any of a simplified molecular-input line-entry system (SMILES) string, MDL Molfile (MDL MOL), Structure Data File (SDF), Protein Data Bank (PDB), Molecule specification file (xyz), International Union of Pure and Applied Chemistry (IUPAC) International Chemical Identifier (InChI), and Tripos Mol2 file (mol2) format. In various embodiments, the labeled training example can include a molecular representation of the small molecule compound, such as a molecular fingerprint or a molecular graph. In various embodiments, the training example can include the small molecule compound expressed in a structure format, which is further converted to a molecular representation (e.g., molecular fingerprint or molecular graph) prior to inputting into the label prediction model.

In various embodiments, the label prediction model is a classifier that predicts the class of inputs. In various embodiments, the label prediction model is trained to generate a binary prediction (e.g., whether a small molecule compound is a likely binder or non-binder). Thus, after training, the label prediction model is evaluated for its ability to accurately predict binders or non-binders according to the assigned labels of the validation dataset. In various embodiments, the label prediction model is trained to generate a multi-class prediction (e.g., a prediction as to whether a small molecule compound is one of a strong binder, weak binder, non-binder, or non-specific binder). Thus, after training, the label prediction model is evaluated for its ability to accurately predict the correct classes according to the assigned labels of the validation dataset. The performance of the label prediction model can be evaluated according to one or more metrics. Example metrics include one or more of a Boltzmann-Enhanced Discrimination of Receiver Operating Curve (BEDROC) metric, an Area Under ROC (AUROC) metric, and an average precision (AVG-PRC) metric. In various embodiments, given N different labels, N different label prediction models are trained and evaluated. Thus, metrics for the N different label prediction models are generated to evaluate the N different label prediction models. In various embodiments, given N different labels, less than N different label prediction models are trained and evaluated. For example, a single label prediction model can be evaluated for its ability to predict N different labels. The single label prediction model is evaluated for its ability to predict the N different labels based on the one or more metrics.

Top-performing labels from amongst the N different labels are selected according to the determined metrics. In various embodiments, the single best performing label is selected from amongst the N different labels. As one example, the single best performing label corresponds to the label prediction model exhibiting the highest metric value. Returning to FIG. 2, the labeled training dataset 220 corresponding to the top-performing labels are selected. Thus, the labeled training dataset 220 can be further used to train the classification model 270. Additionally, given the top-performing labels, the dataset labeling module 140 labels the validation dataset 215 using the top-performing labels to generate a labeled validation dataset 230. The labeled validation dataset 230 can be used to evaluate the classification model 270. Further details of training the classification model 270 are described herein.

Referring to FIG. 3B, it depicts a flow diagram for dataset labeling, in accordance with an embodiment. Step 340 involves labeling the training dataset and/or the validation dataset using a plurality of labels. For example, for a binary classification task, the dataset labeling module 140 labels examples of the training dataset and the validation dataset based on various thresholds.

Step 350 involves evaluating the plurality of labels using label prediction models. As shown in FIG. 3B, step 350 involves steps 355, 360, and 365. Step 355 involves training the label prediction models to predict the labels. At step 360, the label prediction models are validated to determine metrics. Example metrics include one or more of a Boltzmann-Enhanced Discrimination of Receiver Operating Curve (BEDROC) metric, an Area Under ROC (AUROC) metric, and an average precision (AVG-PRC) metric. Here, the metrics are useful for evaluating the labels. As shown in FIG. 3B, the steps of 355 and 360 may be further repeated for different labels. Thus, by repeating steps 355 and 360 over multiple iterations, different metrics for different sets of labels are determined. In various embodiments, iterations of steps 355 and 360 can be performed in parallel. This means that different label prediction models can be trained in parallel and can be validated in parallel.

Step 365 involves selecting the top performing labels based on the determined metrics. The labeled training dataset corresponding to the top performing labels are used to train the classification model (e.g., using supervised learning) and the labeled validation dataset corresponding to the top performing labels can be used to validate the trained classification model.

### Implementing Machine Learning Models

Virtual Screen and Hit Analysis

Disclosed herein are trained machine learning models, such as classification models and/or regression models for conducting a virtual screen or for performing a hit selection and analysis. In various embodiments, a trained classification model is deployed to conduct a virtual screen or perform a hit selection and analysis. In various embodiments, two or more trained classification models are deployed to conduct a virtual screen or perform a hit selection and analysis. In various embodiments, a trained regression model is deployed to conduct a virtual screen or perform a hit selection and analysis. In various embodiments, two or more trained regression models are deployed to conduct a virtual screen or perform a hit selection and analysis. In particular embodiments, three trained regression models are deployed to conduct a virtual screen or perform a hit selection and analysis. Outputs from each of the regression models (e.g., two or regression models, such as three regression models) can be sampled (e.g., equally sampled) to generate a combined total for purposes of conducting the virtual screen or performing a hit selection and analysis. In various embodiments, a trained classification model and a trained regression model are both deployed to conduct a virtual screen or perform a hit selection and analysis. In various embodiments, two or more trained classification models and two or more trained regression models are deployed to conduct a virtual screen or perform a hit selection and analysis.

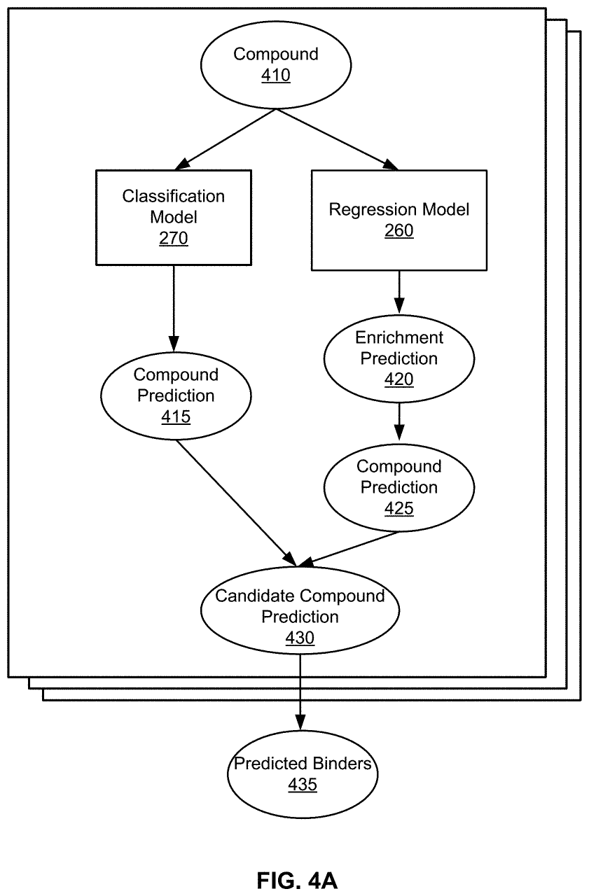

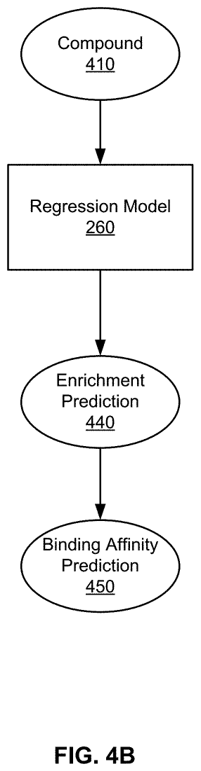

FIG. 4A depicts a flow diagram for deployment of the regression model and/or the classification model for performing a library screen or for identifying hits, in accordance with an embodiment. Generally, the processes shown in FIG. 4A can be performed by one or both of the model deployment module 155 and model output analysis module 160, shown in FIG. 1B. Although FIG. 4A shows the deployment of both the classification model 270 and the regression model 260 for performing a library screen or for identifying hits, in various embodiments, only one of the classification model 270 or the regression model 260 is needed to perform a library screen or to identify hits.

Generally, the flow diagram shown in FIG. 4A can be used to perform a library screen or to identify compound hits that selectively bind to a target. Here, the selectivity arises from the classification model and regression model which have been particularly trained using particular training data or labeled training data. In various embodiments, the target is a binding site. In various embodiments, the target is a nucleic acid, such as a DNA target or a RNA target. In various embodiments, the target is a protein binding site. In various embodiments, the target is a protein-protein interface. For example, the flow diagram shown in FIG. 4A enables identification of compounds that bind a first protein and bind a second protein at a protein-protein interface. Thus, these compounds can be useful for disrupting protein-protein interactions and/or for bringing two proteins within close proximity to one another.

The flow diagram begins with a compound 410. Here, the compound 410 may be an electronic representation of the compound 410. In various embodiments, a compound 410 can be a known compound structure. For example, the compound 410 can be a known compound structure of a DEL. In various embodiments, a compound 410 can be a theoretical product that has not yet been synthesized. In various embodiments, the compound 410 can be a mixture, such as a mixture of building blocks (e.g., synthons) that has not yet been synthesized. In various embodiments, the model deployment module 155 converts the structure format (e.g., any one of simplified molecular-input line-entry system (SMILES) string, MDL Molfile (MDL MOL), Structure Data File (SDF), Protein Data Bank (PDB), Molecule specification file (xyz), International Union of Pure and Applied Chemistry (IUPAC) International Chemical Identifier (InChI), and Tripos Mol2 file (mol2) format) of the compound 410 into molecular representations, such as any of a molecular fingerprint or a molecular graph. Thus, the model deployment module 155 can provide the molecular representation of the compound 410 as input to the classification model 270 and/or the regression model 260.

Referring to the classification model 270, it analyzes the molecular representation of the compound 410 and generates a compound prediction 415. In various embodiments, the compound prediction 415 is a prediction as to whether the compound 410 is likely to bind to a target. In various embodiments, the compound prediction 415 may be a binary value that is indicative of whether the compound 410 is likely to bind to a target. For example, a compound prediction 415 of a value of “1” indicates that the compound is likely to bind to a target. Alternatively a compound prediction 415 of a value of “0” indicates that the compound is unlikely to bind to a target. In various embodiments, the compound prediction 415 may be a value that is indicative of a multi-class designation e.g., whether the compound 410 is a strong binder, a weak binder, a non-binder, or an off target binder.

Referring to the regression model 260, it analyzes the molecular representation of the compound 410 and generates an enrichment prediction 420. Generally, the enrichment prediction 420 is a value that is indicative of binding affinity between the compound 410 and a target. For example, a higher enrichment prediction 420 value is indicative of a higher binding affinity between the compound 410 and the target in comparison to a lower enrichment prediction 420 value.

As shown in FIG. 4A, the enrichment prediction 420 is translated to a compound prediction 425. This step can be performed by the model output analysis module 160. In various embodiments, the compound prediction 425 is an indication that the compound is a likely binder or a likely non-binder to the target. In various embodiments, the model output analysis module 160 categorizes the compound 410 as a likely binder to the target if the enrichment prediction 420 is above a threshold value. In various embodiments, the model output analysis module 160 categorizes the compound 410 as a likely non-binder to the target if the enrichment prediction 420 is below a threshold value. In various embodiments, the compound prediction 425 is an indication that the compound is any of a strong binder, weak binder, non-binder, or non-specific binder.

In various embodiments, the model output analysis module 160 determines a candidate compound prediction 430. In various embodiments, the candidate compound prediction 430 represents overlapping candidate compounds (e.g., overlapping likely binders) predicted by one or more trained models. For example, as shown in the FIG. 4A, the compound prediction 415 from the classification model 270 and the compound prediction 425 derived from the regression model 260 are combined to generate a candidate compound prediction 430. In various embodiments, the candidate compound prediction 430 represents a higher confidence prediction as to whether the compound 410 is a likely binder or non-binder to the target. For example, if both the compound prediction 415 and the compound prediction 425 indicate that the compound 410 is a likely binder, then the candidate compound prediction 430 is a higher confidence prediction that the compound 410 is a likely binder to the target. Conversely, if one or both of the compound prediction 415 and the compound prediction 425 indicate that the compound 410 is a likely non-binder, then the candidate compound prediction 430 is a prediction that the compound 410 is a likely non-binder to the target. Put more generally, the candidate compound prediction 430 indicates that the compound 410 is a likely binder to the target if both the compound prediction 415 and the compound prediction 425 indicate that the compound 410 is a likely binder to the target. Thus, a candidate compound prediction 430 that indicates that the compound 410 is a likely binder to the target represents overlapping candidate compounds predicted to be binders by the classification model and by the regression model.

In various embodiments, such as embodiments in which only one of the classification model 270 or the regression model 260 is deployed, the compound prediction 415 or compound prediction 425 can directly serve as the candidate compound prediction 430. For example, if only the classification model 270 is deployed, the classification model 270 analyzes the representation of the compound 410 and determines a compound prediction 415 that indicates the compound 410 is a likely binder to a target. Thus, the compound prediction 415 can serve as the candidate compound prediction 430 and therefore, the compound 410 can be selected as a candidate compound (e.g., a compound that is a likely binder). As another example, if only the regression model 260 is deployed, the regression model 260 analyzes the representation of the compound 410 and determines an enrichment prediction 420 that can be further transformed to the compound prediction 425 that indicates the compound 410 is a likely binder to a target. Here, the compound prediction 425 serves as the candidate compound prediction 430 and the compound 410 can be selected as a candidate compound (e.g., a compound that is a likely binder).

In various embodiments, the candidate compound prediction 430 represents overlapping candidate compounds (e.g., overlapping likely binders) predicted by multiple classification models or predicted by multiple regression models 260. For example, multiple classification models can be differently trained to predict likely binders to the same or similar targets. Thus, two or more classification models can be deployed to generate compound predictions (e.g., compound prediction 415 shown in FIG. 4A). Therefore, the candidate compound prediction 430 identifies overlapping binders predicted by each of the two or more classification models. As another example, multiple regression models can be differently trained to predict values indicative of binding affinity between compounds and the same or similar targets. In various embodiments, two or more regression models can be deployed to generate two or more enrichment predictions (e.g., enrichment prediction 420 shown in FIG. 4A). The two or more enrichment predictions 420 can be transformed to two or more compound predictions 425 and therefore, the candidate compound prediction 430 identifies overlapping binders predicted by each of the two or more regression models 260. In particular embodiments, three regression models are deployed to generate three enrichment predictions (e.g., enrichment prediction 420 shown in FIG. 4A) that can be transformed to three compound predictions 430.

Altogether, the process described above refers to determination of a candidate compound prediction 430 for a single compound 410. The process can be repeated for additional compounds. For example, the process can be repeated for other compounds in a library, such as a virtual library (e.g., a virtual DEL). Thus, individual candidate compound predictions 430 can be determined for compounds in the virtual library and predicted binders 435 across the full virtual library can be identified according to the candidate compound predictions 430. Here, the predicted binders 435 represent the set of compounds that are likely binders to the target identified through the virtual library screen. In various embodiments, the predicted binders 435 refer to compound hits that are predicted to bind to the target.

In various embodiments, the predicted binders 435 refer to building blocks of compounds that are predicted to influence binding of a compound to the target. For example, predicted binders 435 can be individual synthons that contribute to specific binding between candidate compounds that include one or more of the synthons and the target. Thus, the individual synthons that are predicted to contribute towards binding to a target can be further included in additional compounds for testing against the target. In various embodiments, instead of predicted binders 435, as is shown in FIG. 4A, predicted non-binders can also be determined (not shown in FIG. 4A). Specifically, predicted non-binders can refer to compounds that are unlikely to bind to the target. As another example, predicted non-binders can refer to building blocks of compounds, such as synthons, that are predicted to negatively influence the binding of a compound to the target. Thus, such synthons that negatively influence the binding of a compound to the target can be identified and removed to ensure that future compounds that are tested against the target do not include those synthons.

In various embodiments, based on the candidate compounds whose candidate compound prediction 430 indicates that they are likely binders to a target, the predicted binders 435 are determined by performing a clustering methodology to obtain chemical diversity across the candidate compounds. Thus, a subset of the candidate compounds can be selected for synthesis and further testing (e.g., synthesis and in vitro testing against the target). For example, the candidate compounds (e.g., compounds whose candidate compound prediction 430 indicate that they are likely binders to a target) are clustered according to the similarity of their structures. For example, the similarity of structures between candidate compounds can be calculated according to similarities of the molecular representations of the candidate compounds. In particular embodiments, the similarity of structures between candidate compounds is calculated via Jaccard similarity of molecular fingerprints (e.g., Morgan fingerprints) of the candidate compounds. Thus, the candidate compounds can be clustered using an unsupervised clustering methodology (e.g., Taylor-Butina clustering).

In various embodiments, candidate compounds can be assigned to two or more clusters. In various embodiments, candidate compounds can be assigned to three or more clusters, four or more clusters, five or more clusters, six or more clusters, seven or more clusters, eight or more clusters, nine or more clusters, ten or more clusters, eleven or more clusters, twelve or more clusters, thirteen or more clusters, fourteen or more clusters, fifteen or more clusters, sixteen or more clusters, seventeen or more clusters, eighteen or more clusters, nineteen or more clusters, twenty or more clusters, twenty one or more clusters, twenty two or more clusters, twenty three or more clusters, twenty four or more clusters, twenty five or more clusters, twenty six or more clusters, twenty seven or more clusters, twenty eight or more clusters, twenty nine or more clusters, or thirty or more clusters. In particular embodiments, candidate compounds can be assigned to 26 or more clusters. This ensures that candidate compounds from different clusters are structurally diverse.

In various embodiments, the predicted binders 435 are a subset of the candidate compounds assigned to the different clusters. In various embodiments, the predicted binders 435 can include one or more compounds from each of the different clusters. In particular embodiments, the predicted binders 435 include one compound from each cluster. For example, if the candidate compounds were clustered into 10 clusters, the predicted binders 435 include 10 candidate compounds, one compound selected from each of the 10 clusters. Thus, the 10 predicted binders 435 are structurally diverse and can undergo subsequent testing (e.g., synthesis and in vitro testing against the target).

Predicting Binding Affinity

In various embodiments, a trained regression model is deployed to predict a value that is indicative of binding affinity between compounds and targets. The regression model is able to predict a continuous value that is indicative of binding affinity and therefore, is implemented for predicting binding affinity between compound and targets. As described herein, the value indicative of binding affinity can be an enrichment prediction that is correlated with binding affinity. Generally, the enrichment prediction represents a de-noised and de-biased prediction absent the effects of covariates.

Referring to FIG. 4B, it depicts a flow diagram for predicting binding affinity using a regression model, in accordance with an embodiment. Generally, the flow diagram shown in FIG. 4B can be used to predict binding affinity between compounds and targets. In various embodiments, the target is a binding site. In various embodiments, the target is a protein binding site. In various embodiments, the target is a protein-protein interface. For example, the flow diagram shown in FIG. 4B predicts binding affinity of a compound that may bind a first protein and bind a second protein at a protein-protein interface.

The flow diagram in FIG. 4B begins with a compound 410. Here, the compound 410 may be an electronic structure format of the compound 410. In various embodiments, a compound 410 can be a known compound structure. For example, the compound 410 can be a known compound structure in a DEL. In various embodiments, a compound 410 can be a theoretical product that has not yet been synthesized. In various embodiments, the compound 410 can be a mixture, such as a mixture of building blocks (e.g., synthons) that has not yet been synthesized. In various embodiments, the model deployment module 155 converts the structure format of the compound 410 into molecular representations, such as any of a molecular fingerprint or a molecular graph. Thus, the model deployment module 155 can provide the molecular representation of the compound 410 as input to the regression model 260.

The regression model 260 generates an enrichment prediction 440, which is a value indicative of binding affinity. Generally, a higher enrichment prediction 440 value is indicative of a higher binding affinity between the compound 410 and the target in comparison to a lower enrichment prediction 440 value. The regression model 260 leverages negative control data to correct noise from non-target interactions in the data from the target screen. Further description of the regression model 260 and its structure and functionality is described herein.

As shown in FIG. 4B, the enrichment prediction 440 is converted to a binding affinity prediction 450. In various embodiments, the binding affinity prediction 450 is a binding affinity value. In various embodiments, a binding affinity value is measured by an equilibrium dissociation constant (K_{d}). In various embodiments, a binding affinity value is measured by the negative log value of the equilibrium dissociation constant (pK_{d}). In various embodiments, a binding affinity value is measured by an equilibrium inhibition constant (Kᵢ). In various embodiments, a binding affinity value is measured by the negative log value of the equilibrium inhibition constant (pKᵢ). In various embodiments, a binding affinity value is measured by the half maximal inhibitory concentration value (IC50). In various embodiments, a binding affinity value is measured by the half maximal effective concentration value (EC50). In various embodiments, a binding affinity value is measured by the equilibrium association constant (Kₐ). In various embodiments, a binding affinity value is measured by the negative log value of the equilibrium association constant (Kₐ). In various embodiments, a binding affinity value is measured by a percent activation value. In various embodiments, a binding affinity value is measured by a percent inhibition value.

In various embodiments, the enrichment prediction 440 is converted to a binding affinity prediction 450 according to a pre-determined conversion relationship. The pre-determined conversion relationship may be determined using DEL experimental data such as previously generated DEL outputs (e.g., DEL output 120A and 120B shown in FIG. 1A) based on DEL experiments. In various embodiments, the pre-determined conversion relationship is a linear equation. Here, the enrichment prediction 440 is linearly correlated to the binding affinity prediction 450. In various embodiments, the pre-determined conversion relationship is any of an exponential, logarithmic, non-linear, or polynomial equation.

Generally, in a medicinal chemistry campaign such as hit-to-lead optimization, binding affinity predictions are commonly used to assess and select the next compounds to be synthesized. The regression model 260 disclosed herein enables the rank ordering and binding affinity predictions useful for this task and can hence be used directly to guide design. Additionally the fine grained interpretation of contributions to the binding is useful for design. This methodology has the major advantage of being able to create a regression model 260 right after screening for the hit-to-lead optimization compared to the classical pipeline. Usually, machine learned models are only generated once many compounds have been synthesized and assayed which takes several months to years after the initial screening that identified the hit. Additionally, a more focused DEL could be synthesized to create an appropriate regression model. In particular, the analysis of the structure-binding relationship from the regression model can help the selection of synthons to be incorporated in the next library design.

### Example Machine Learning Models

Embodiments disclosed herein involve training and/or deploying machine learning models for generating predictions for any of a virtual screen, hit selection and analysis, or predicting binding affinity. In various embodiments, machine learning models disclosed herein can be any one of a regression model (e.g., linear regression, logistic regression, or polynomial regression), decision tree, random forest, support vector machine, Naïve Bayes model, k-means cluster, or neural network (e.g., feed-forward networks, convolutional neural networks (CNN), deep neural networks (DNN), autoencoder neural networks, generative adversarial networks, attention based models, or recurrent networks (e.g., long short-term memory networks (LSTM), bi-directional recurrent networks, deep bi-directional recurrent networks).

In various embodiments, machine learning models disclosed herein can be trained using a machine learning implemented method, such as any one of a linear regression algorithm, logistic regression algorithm, decision tree algorithm, support vector machine classification, Naïve Bayes classification, K-Nearest Neighbor classification, random forest algorithm, deep learning algorithm, gradient boosting algorithm, gradient based optimization technique, and dimensionality reduction techniques such as manifold learning, principal component analysis, factor analysis, autoencoder, and independent component analysis, or combinations thereof. In various embodiments, the machine learning model is trained using supervised learning algorithms, unsupervised learning algorithms, semi-supervised learning algorithms (e.g., partial supervision), weak supervision, transfer, multi-task learning, or any combination thereof.

In various embodiments, machine learning models disclosed herein have one or more parameters, such as hyperparameters or model parameters. Hyperparameters are generally established prior to training. Examples of hyperparameters include the learning rate, depth or leaves of a decision tree, number of hidden layers in a deep neural network, number of clusters in a k-means cluster, penalty in a regression model, and a regularization parameter associated with a cost function. As described in further detail herein, machine learning models may include an augmentation hyperparameter that can control the implementation of one or more augmentations. An augmentation hyperparameter may be a probability value that is tuned prior to training. Model parameters are generally adjusted during training. Examples of model parameters include weights associated with nodes in layers of a neural network, support vectors in a support vector machine, and coefficients in a regression model. The model parameters of the machine learning model are trained (e.g., adjusted) using the training data to improve the predictive power of the machine learning model.

In particular embodiments, an example machine learning model is a regression model. Generally, a regression model analyzes a compound (e.g., analyzes a representation of the compound) and generates a prediction value that is useful for a virtual screen, hit selection and analysis, or predicting binding affinity. In various embodiments, the prediction value is a value on a continuous scale. In various embodiments, the prediction value is a multi-classification value. In various embodiments, the prediction value is a binary value. In particular embodiments, the regression model generates an enrichment prediction that is indicative of binding affinity between the compound and a target of interest.

In particular embodiments, the regression model is structured to incorporate and separate the effects of one or more covariates. Therefore, the enrichment prediction generated by the regression model can represent a denoised or debiased value that avoids the effects of the one or more covariates. Example covariates include, without limitation, non-target specific binding (e.g., binding to beads, binding to streptavidin of the beads, binding to biotin, binding to gels, binding to DEL container surfaces, binding to tags e.g., DNA tags or protein tags), enrichment in other negative control pans, compound synthesis yield, reaction type, starting tag imbalance, initial load populations, experimental conditions, chemical reaction yields, side and truncated products, errors from the library synthesis, DNA affinity to target, sequencing depth, and sequencing noise such as PCR bias. In particular embodiments, the regression model incorporates effects of at least two covariates. In particular embodiments, the regression model incorporates effects of at least three covariates, at least four covariates, at least five covariates, at least six covariates, at least seven covariates, at least eight covariates, at least nine covariates, at least ten covariates, at least eleven covariates, at least twelve covariates, at least thirteen covariates, at least fourteen covariates, at least fifteen covariates, at least sixteen covariates, at least seventeen covariates, at least eighteen covariates, at least nineteen covariates, or at least twenty covariates.

Generally, the selection of hits on a DEL selection suffers from the need to consider the effects of various covariates when rank ordering binders in order to select strong binders and avoid selection of non-specific or promiscuous binders. The regression model implicitly performs this denoising, because predicting these covariates is incorporated into the learning objective. As a result, the predictions provided by the regression model provide a better estimate of binding affinity which has noise and non-specific affinity removed from it. This denoising means that the regression model provides a better rank ordering of compounds by their binding affinity than could be obtained from a simple score, such as enrichment over the tag imbalance or over a negative control. In some scenarios, the regression model can provide a more fine grained detail on contributions of building blocks, including synthons, contributing to specific and non-specific/promiscuous binding. This enables a better understanding of the structure-binding relationship and could be used to identify non-specific/promiscuous synthons to be avoided in future libraries.

In various embodiments, the regression model is structured to incorporate the effects of one or more covariates, and is further structured to generate predictions of two or more targets (e.g., protein targets) of interest. For example, the regression model is trained via multi-task learning and therefore, is structured to generate multiple predictions. Here, training a regression model via multi-task learning to generate predictions for two or more targets can be beneficial, because 1) training jointly may help to regularize the model to improve its generalizability, and 2) information of the different targets (e.g., protein targets) can be shared such that the regression model can generate improved predictions for each of the two or more targets.

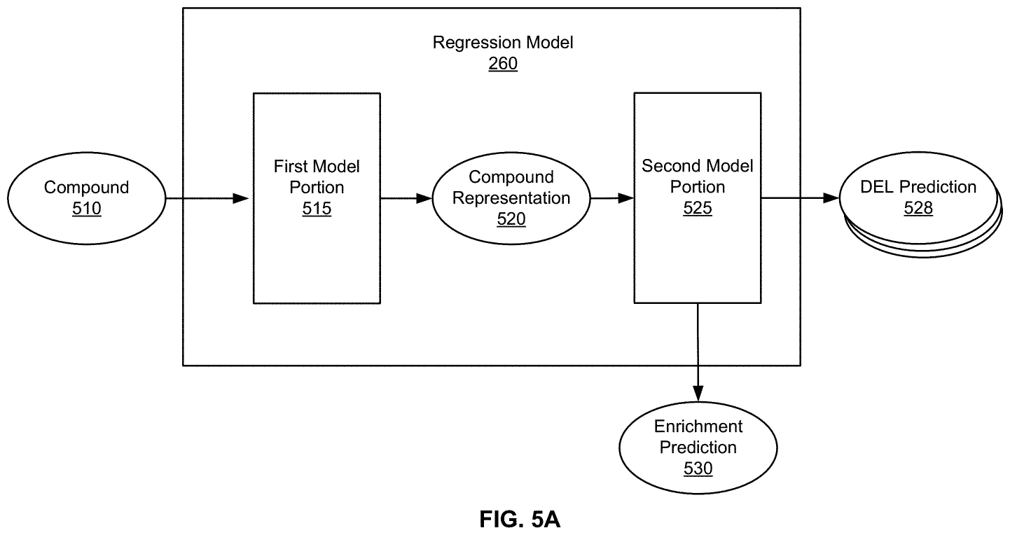

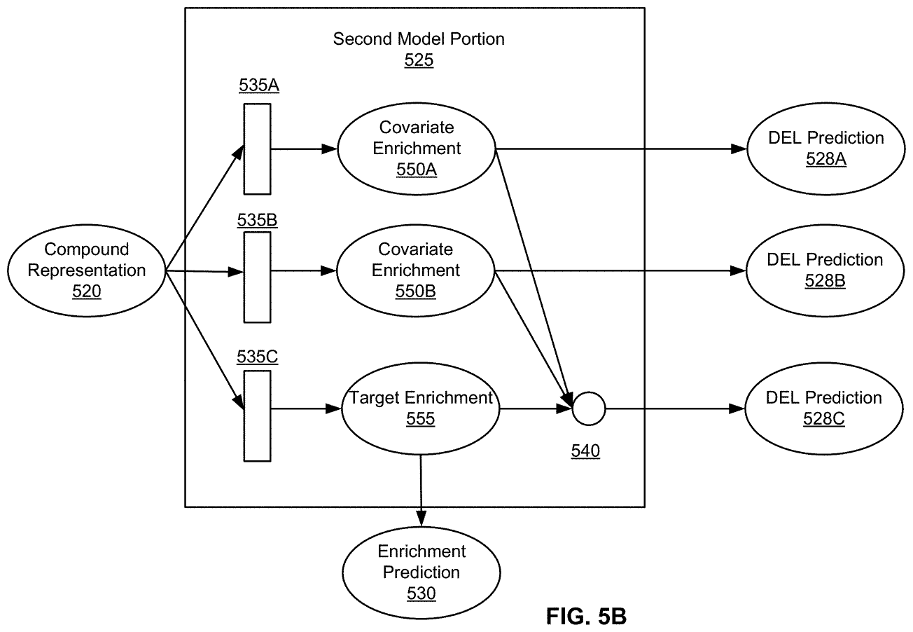

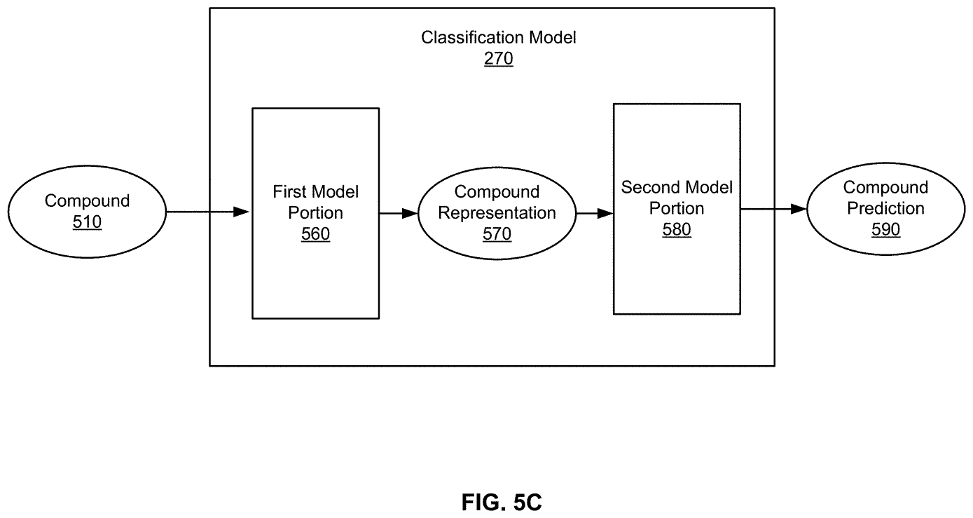

Reference is now made to FIG. 5A, which depicts an example structure of a regression model, in accordance with an embodiment. As shown in FIG. 5A, the regression model 260 receives, as input, the compound 510 (e.g., a representation of the compound 510) and generates an enrichment prediction 530 and one or more DEL prediction 528. For example, the compound 510 can be represented as a molecular fingerprint or a molecular graph and provided as input to the regression model 260. The regression model 260 can include a first model portion 515 and a second model portion 525. As shown in FIG. 5A, the first model portion 515 analyzes the compound 510 and outputs a transformed compound representation 520. The transformed compound representation 520 is provided as input to the second model portion 525, which then generates the enrichment prediction 530. Here, the enrichment prediction 530 represents the de-noised and de-biased value that is absent effects of covariates. As shown in FIG. 5A, the second model portion 525 may further output one or more DEL predictions 528. Here a DEL prediction 528 can be a predicted DEL count, such as a UMI count.

Generally, the first model portion 515 translates a representation of compound 510 to a compound representation 520 with fixed dimensionality. In various embodiments, the first model portion 515 translates the compound 510 to a compound representation 520 of higher dimensionality. In various embodiments, the first model portion 515 translates the compound 510 to a compound representation 520 of a lower dimensionality. In various embodiments, the compound 510 can be a 1×N vector representation. Here, N can be greater than 500, greater than 750, greater than 1000, greater than 2000, greater than 3000, greater than 4000, greater than 5000, greater than 6000, greater than 7000, greater than 8000, greater than 9000, or greater than 10,000. Thus, the transformed compound representation 520 may be a 1×M vector representation. In various embodiments, M is greater than N. In various embodiments, M is the same as N. In various embodiments, M is less than N. Here, the transformed compound representation 520 can be referred to as an embedding. In particular embodiments, M is less than 500. In particular embodiments, M is less than 400. In particular embodiments, M is less than 300. In particular embodiments, M is less than 200. In particular embodiments, M is less than 100.

In various embodiments, the compound 510 can be a molecular graph representation which can include multiple tensors. In various embodiments, tensors can include a node feature matrix capturing atom features such as number of atoms in the compound and location of atoms in the compound. In various embodiments, tensors can include an adjacency/bond matrix that describes relationships between atoms of the compound and bond characteristics of the compound. In various embodiments, tensors can include 3D locations. In various embodiments, tensors can include a distance matrix. Here, the first model portion 515 translates the dimensionality of the molecular graph representation to achieve a transformed compound representation 520 with lower dimensionality in comparison to the molecular graph representation. For example, the transformed compound representation 520 may be a P×Q representation of lower dimensionality in comparison to the molecular graph representation (e.g., P and Q are less than the corresponding dimensionality values of the molecular graph representation). In particular embodiments, P is 1 and therefore, the transformed compound representation 520 is a 1×Q vector representation. In particular embodiments, Q is less than 500. In particular embodiments, Q is less than 400. In particular embodiments, Q is less than 300. In particular embodiments, Q is less than 200. In particular embodiments, Q is less than 100.

In various embodiments, the first model portion 515 is a learned network. In various embodiments, the first model portion 515 may be a neural network. In various embodiments, the first model portion 515 may be a graph neural network. In various embodiments, the first model portion 515 may be an encoder network. In various embodiments, the first model portion 515 may be a GIN-E encoder. In various embodiments, the first model portion 515 may be an attention based model. In various embodiments, the first model portion 515 may be a multilayer perceptron.

In various embodiments, the first model portion 515 is not a trainable network. For example, the first model portion 515 may transform the compound 510 to a transformed compound representation 520 of lower dimensionality through fixed processes (e.g., non-learned processes). In various embodiments, the transformed compound representation 520 is a Morgan fingerprint representation.

Reference is now made to FIG. 5B, which depicts an example second model portion 525 of the regression model 260, in accordance with the embodiment shown in FIG. 5A. Here, the second model portion 525 receives the transformed compound representation 520 as input and determines the enrichment prediction 530. Generally, the second model portion 525 includes multiple heads or paths that each predict an enrichment value based on the transformed compound representation 520. Here, each enrichment value represents an intermediate value. The final value in each head is a DEL prediction (e.g., DEL prediction 528A, DEL prediction 528B, and DEL prediction 528C). In various embodiments, each DEL prediction represents one of DEL counts, DEL reads, or DEL indices for a modeled experiment. At least one of the heads represents a modeled experiment which is designed to elucidate and enable incorporation of the effects of a covariate. In particular embodiments, the second model portion 525 includes at least two heads representing two modeled experiments that are designed to elucidate and enable incorporation of the effects of at least two covariates. Although FIG. 5B shows three total heads (e.g., two that lead to a covariate enrichment, and one that leads to a target enrichment), in other embodiments there may be additional or fewer heads. For example, there may be two heads (e.g., one leading to a covariate enrichment, and one leading to a target enrichment). As another example, there may be N heads, where N−1 heads lead to covariate enrichments and one leads to a target enrichment. Altogether, the regression model 260 is structured such that as it is trained, it improves the accuracy of predicting the DEL predictions (e.g., 528A, 528B, and/or 528C) which represent predictions for experimental DEL counts, DEL reads, or DEL indices. Additionally, the intermediate covariate enrichment values (e.g., 550A and 550B) accurately represent the effects of corresponding covariates whereas the target enrichment value 555 accurately represents the de-noised and de-biased value that is absent the effects of the covariates.

As a specific example, assume a second model portion 525 that includes two heads that model two different experiments. A first modeled experiment refers to bead mounted target proteins that are exposed to DEL compounds. A second modeled experiment refers to beads (absent target proteins) that are exposed to DEL compounds. Therefore, a first head of the second model portion 525 generates a target enrichment (e.g., target enrichment value 555) for the first modeled experiment. Here, the target enrichment value represents a value absent the effects of one or more covariates, such as the covariate of DEL compounds that bind to beads (as opposed to target proteins).

The second head of the second model portion 525 generates a covariate enrichment for the second modeled experiment. In various embodiments, the second model portion 525 can include additional heads for modeling additional experiments to quantify signals arising from other covariates, thereby enabling the determination of an improved signal that is arising mainly from specific target protein and compound binding. For example, the second model portion 525 can include an additional head for modeling an additional experiment to quantify signals arising from an additional covariate, such as the covariate of a small molecule compound binding to linkers (e.g., streptavidin linkers) on beads. In this example, the second model portion 525 models a first experiment of binding between small molecule compounds and bead mounted target proteins, a second experiment of binding between small molecule compounds and beads, and a third experiment of binding between small molecule compounds and linkers on beads. In various embodiments, the second model portion 525 can include yet further additional heads for modeling additional covariates (e.g., a fourth head for modeling off target binding e.g., to another protein).

In various embodiments, the regression model is structured to generate predictions of two or more targets (e.g., protein targets) of interest. For example, the regression model is trained via multi-task learning and therefore, is structured to generate multiple predictions. In such embodiments, the regression model includes a head or path for each target of interest. For example, given two targets (e.g., protein targets) of interest, the regression model includes two enrichment heads (one for each target) and each of those heads will receive information about shared or separate set of covariates enrichments.

In the embodiment shown in FIG. 5B, the second model portion 525 includes three heads, a first head including a layer 535C that generates a target enrichment 555 and a corresponding DEL prediction 528C, a second head including a layer 535A that generates a covariate enrichment 550A and a corresponding DEL prediction 528A, and a third head including a layer 535B that generates a covariate enrichment 550B and a corresponding DEL prediction 528B. As shown in FIG. 5B. The target enrichment 555, covariate enrichment 550A, and covariate enrichment 550B are combined 540 to generate the DEL prediction 528C. Furthermore, the target enrichment 555 value is taken as the enrichment prediction 530. Here, the enrichment prediction 530 represents the de-noised and de-biased value that can be used for performing any of the virtual screen, identifying hits, and predicting binding affinity, as is described herein.

In various embodiments, the second model portion 525 can include fewer or additional heads. For example, the second model portion 525 may only include a first head including a layer 535C that generates a target enrichment 555 and a second head including a layer 535A that generates covariate enrichment 550A. Thus, the target enrichment 555 and covariate enrichment 550A are combined 540 to generate the DEL prediction 528C. As another example, the second model portion 525 may include N heads, where one of the heads generates a target enrichment value (e.g., target enrichment 555) and the other N−1 heads generate covariate enrichments (e.g., covariate enrichment 550A and 550B). Thus, the target enrichment 555 and the N−1 covariate enrichments can be combined to generate the DEL prediction. In other words, the second model portion 525 incorporates the effects of the N−1 different covariates. In various embodiments, N can be any of 4, 5, 6, 7, 8, 9, 10, 11, 12, 13, 14, 15, 16, 17, 18, 19, or 20. In particular embodiments, N is 14. Thus, the second model portion 525 incorporates the effects of 13 different covariates. In various embodiments, a covariate enrichment (e.g., covariate enrichment 550A or covariate enrichment 550B) can represent the effects from two or more covariates. For example, covariate enrichment 550A can correspond to a modeled experiment that models the effects of the two covariates of 1) negative pan enrichment and 2) load count. Thus, there need not be a 1 to 1 relationship between the number of heads in the second model portion 525 and the number of covariates.

Referring to the layers 535A, 535B, and 535C each layer reduces the dimensionality of the transformed compound representation 520 to a lower dimensional value (e.g., target enrichment 555, covariate enrichment 550A, and covariate enrichment 550B). In various embodiments, each of the target enrichment 555, covariate enrichment 550A, and covariate enrichment 550B are single float values (e.g., one dimension). Therefore, each layer 535A, 535B, and 535C reduces the transformed compound representation 520 to single dimensional float values. In various embodiments, although not shown in FIG. 5B, the second model portion 525 may further include one or more preceding layers situated between the layers 535A, 535B, and 535C and transformed compound representation 520. Therefore, the one or more preceding layers may translate the dimensionality of the transformed compound representation 520 to an intermediate representation, and the intermediate representation can be provided to each of the layers 535A, 535B, and 535C. In various embodiments, the one or more preceding layers include a rectified linear unit (ReLu). In particular embodiments, the transformed compound representation 520 is a 1×300 dimensional vector. The one or more preceding layers reduces the transformed compound representation 520 to a 1×128 dimensional vector. The layers 535A, 535B, and 535C reduce the 1×128 dimensional vector to single dimensional float values.

Generally, at 540, the target enrichment 555 is combined with the different covariate enrichments (e.g., covariate enrichment 550A and 550B) using learned parameters to generate the DEL prediction 528C. In one embodiment, the DEL prediction 528C can be calculated in Equation 1 as:

DEL prediction=(X+β₁Y₁+β₂Y₂+ . . . +βₙYₙ+βₙ₊₁) (1)

where X is the target enrichment 555, β₁, β₂ . . . βₙ₊₁ are learned parameters of the regression model, and each of Y₁, Y₂ . . . Yₙ represents a covariate enrichment (e.g., covariate enrichment 550A and 550B).

In some embodiments, the DEL prediction 528C is generated by combining the target enrichment 555, the covariate enrichments (e.g., covariate enrichment 550A and 550B), and an observed load count (e.g., population of molecules at the start of an experiment e.g., DEL experiment). For example, the DEL prediction 528C can be calculated in Equation 2 as:

DEL prediction=(X+β₁*f(Y₁,Y₂. . . Yₙ)+β₂*Z+β₃) (2)

where X is the target enrichment 555, β₁, β₂ and β₃ are learned parameters of the regression model, f is a given function, each of Y₁, Y₂ . . . Yₙ represents a covariate enrichment (e.g., covariate enrichment 550A and 550B), and Z represents the observed load count. In various embodiments, f is a non-linear function. In various embodiments, f(Y₁, Y₂ . . . Yₙ) represents max (Y₁, Y₂ . . . Yₙ). In various embodiments, f(Y₁, Y₂ . . . Yₙ) represents sum (Y₁, Y₂ . . . Yₙ). In various embodiments, f(Y₁, Y₂ . . . Yₙ) represents polynomial (Y₁, Y₂ . . . Yₙ).

In various embodiments, each of the heads or paths of the second model portion 525 terminates in a DEL prediction 528 (e.g., DEL count such as UMI count), with the covariate enrichment (e.g., covariate enrichment 550A or 550B) or target enrichment (e.g., target enrichment 555) serving an intermediate value. For example, for the first head or path of the second model portion 525, the covariate enrichment 550A is an intermediate value for calculating a DEL prediction, 528A. The DEL prediction 528A of the first head, referred to as DEL Prediction, can be calculated in Equation 3 as:

DEL Prediction₁=Y₁+α₁Z+α₂ (3)

where Y₁ represents covariate enrichment 550A, Z is the observed load count, and α₁ and α₂ are learnable parameters of the regression model.

As another example, for the second head or path of the second model portion 525, the covariate enrichment 550B is an intermediate value for calculating a DEL prediction 528B. The DEL prediction 528B of the second head, referred to as DEL Prediction₂, can be calculated in Equation 4 as:

DEL Prediction₂=Y₂+α₃Z+α₄ (4)

where Y₂ represents covariate enrichment 550B, Z is the observed load count, and α₃ and α₄ are learnable parameters of the regression model.

In various embodiments, the second model portion 525 is structured to generate predictions for two or more targets (e.g., protein targets) of interest. In such embodiments, the regression model includes a head or path for each target of interest. For example, returning to FIG. 5B, it shows one head for a target enrichment 555 which is specific for a protein of interest. Therefore, for a second target of interest, the second model portion 525 can further include an additional head for a second target enrichment that is specific for a second target of interest. Thus, the second target enrichment can be combined with covariate enrichments (e.g., covariate enrichment 550A and 550B) to generate a DEL prediction for the second target of interest.

Although FIG. 5B shows the second model portion 525 of a single regression model 260, in various embodiments, more than a single regression model 260 can be implemented. In various embodiments, two or more regression models can be implemented. In particular embodiments, three regression models can be implemented. Here, the regression models may be implemented in parallel. Each of the regression models (e.g., three regression models) may include a second model portion 525 as shown in FIG. 5B, and therefore, each regression model can be configured to generate a target enrichment value (e.g., target enrichment 555). In various embodiments, the target enrichment value from each of the regression models can be combined to generate the enrichment prediction 530. For example, the target enrichment value from each of the regression models can be statistically combined (e.g., mean, median, or nth percentile).

In various embodiments, each of the target enrichment values can be used to parameterize a distribution. In some embodiments, the distribution is a Poisson distribution. In some embodiments, the distribution is a negative binomial distribution. For example, a negative distribution may include two parameters, where a first parameter is the target enrichment value. The second parameter may be a scalar constant, herein referred to as a. In such embodiments, a mixture sampled from the individual distributions can be generated and statistical measures (e.g., mean, median, or nth percentile) of the mixture can be determined. For example, in a scenario involving implementation of three regression models, a mixture may be equally sampled from three individual distributions (e.g., negative binomial distributions). Taking a statistical measure as an enrichment prediction 530, the enrichment prediction 530 value can be used for performing any of the virtual screen, identifying hits, and predicting binding affinity, as is described herein.

In particular embodiments, an example machine learning model is a classification model. Generally, a classification model analyzes a compound (e.g., analyzes a representation of the compound) and generates a prediction that is useful for a virtual screen or for a hit selection and analysis. In various embodiments, the prediction is a binary prediction for the compound. For example, the prediction can be indicative of whether the compound is predicted to bind to a target or predicted to not bind to a target. For example, a prediction of a value of “1” can indicate that the compound is predicted to bind to a target. A prediction of a value of “0” can indicate that the compound is predicted to not bind to a target.

FIG. 5C depicts an example structure of a classification model 270, in accordance with an embodiment. As shown in FIG. 5A, the classification model 270 receives, as input, the compound 510 (e.g., a representation of the compound 510) and generates a compound prediction 590. For example, the compound 510 can be represented as a molecular fingerprint or a molecular graph and provided as input to the classification model 270. The classification model 270 can include a first model portion 560 and a second model portion 580. As shown in FIG. 5C, the first model portion 560 analyzes the compound 510 and outputs a transformed compound representation 570. The transformed compound representation 570 is provided as input to the second model portion 560, which then generates the compound prediction 590. In various embodiments, the transformed compound representation 570 is an embedding.

Generally, the first model portion 560 reduces the dimensionality of the compound 510 to a transformed compound representation 570 of a lower dimensionality. In various embodiments, the compound 510 can be a 1×V vector representation. Here, V can be greater than 500, greater than 750, greater than 1000, greater than 2000, greater than 3000, greater than 4000, greater than 5000, greater than 6000, greater than 7000, greater than 8000, greater than 9000, or greater than 10,000. Thus, the transformed compound representation 520 may be a 1×W vector representation of lower dimensionality (e.g., W is less than V). In particular embodiments, W is less than 500. In particular embodiments, W is less than 400. In particular embodiments, W is less than 300. In particular embodiments, W is less than 200. In particular embodiments, W is less than 100.

In various embodiments, the compound 510 can be a molecular graph representation which can include multiple tensors. Tensors can include a node feature matrix capturing atom features such as number of atoms in the compound and location of atoms in the compound. Tensors can also include an adjacency/bond matrix that describes relationships between atoms of the compound and bond characteristics of the compound. Here, the first model portion 560 reduces the dimensionality of the molecular graph representation to achieve a transformed compound representation 570 with lower dimensionality in comparison to the molecular graph representation. For example, the transformed compound representation 570 may be a R×S representation of lower dimensionality in comparison to the molecular graph representation (e.g., R and S are less than the corresponding dimensionality values of the molecular graph representation). In particular embodiments, R is 1 and therefore, the transformed compound representation 570 is a 1×S vector representation. In particular embodiments, S is less than 500. In particular embodiments, S is less than 400. In particular embodiments, S is less than 300. In particular embodiments, S is less than 200. In particular embodiments, S is less than 100.

In various embodiments, the first model portion 560 of the classification model 270 is the same as the first model portion 515 of the regression model 260 (see FIG. 5A). In various embodiments, the first model portion 560 of the classification model 270 is different from the first model portion 515 of the regression model 260 (see FIG. 5A). In various embodiments, the first model portion 560 is a learned network. In various embodiments, the first model portion 560 may be a neural network. In various embodiments, the first model portion 560 may be a graph neural network. In various embodiments, the first model portion 560 may be an encoder network. In various embodiments, the first model portion 560 may be a GIN-E encoder. In various embodiments, the first model portion 560 may be an attention based model. In various embodiments, the first model portion 560 may be a multilayer perceptron.

In various embodiments, the first model portion 560 is not a trainable network. For example, the first model portion 560 may transform the compound 510 to a transformed compound representation 570 of lower dimensionality through fixed processes (e.g., non-learned processes). In various embodiments, the transformed compound representation 560 is any of a RDKit fingerprint representation, RDKit layered fingerprint representation, Avalon fingerprint representation, Atom-Pair and Topological Torsion fingerprint representation, 2D Pharmacophore fingerprint representation, or a Morgan fingerprint representation.

Referring next to the second model portion 580 of the classification model 270, it analyzes the transformed compound representation 570 and generates a compound prediction. Generally, the second model portion 580 of the classification model 270 is different from the second model portion 525 of the regression model 260 (see FIG. 5A).

The second model portion 580 includes one or more layers for reducing the dimensionality of the transformed compound representation 570 to the compound prediction 590. Here, the compound prediction 590 can be a single dimensional float value. In various embodiments, the second model portion 580 includes a rectified linear unit (ReLu). In particular embodiments, the transformed compound representation 570 is a 1×300 dimensional vector. The second model portion 580 reduces the transformed compound representation 580 to the single dimensional float value of the compound prediction 590.

Although embodiments disclosed herein describe classification models and regression models as separate machine learning models, in various embodiments, a single model can embody both the classification model and the regression model. For example, a single model can analyze a molecular representation of a compound and output two predictions: 1) a binary prediction of whether the compound is a likely binder or a non-binder to the target and 2) a continuous value DEL prediction that is indicative of the binding affinity between the compound and the target. Thus, the single model can be deployed for conducting a virtual screen, for predicting hits, and for predicting binding affinity.

In such embodiments where the single model embodies both the classification model and the regression model, the structure of the single model may include a portion that is shared between the classification model and the regression model. For example, referring again to FIGS. 5A and 5C, the first model portion 515 of the regression model 260 and the first model portion 560 of the classification model 270 may be shared whereas the second model portion 525 or the regression model 260 and second model portion 580 of the classification model 270 may be separate. Therefore, the compound representation 520 or compound representation 570 may be the same representation and the single model need only generate the representation once. Therefore, the single model analyzes a molecular representation of a compound 510, reduces the dimensionality of the molecular representation of the compound 510 to the compound representation (e.g., compound representation 520 or 570) using a first model portion (e.g., first model portion 515 or 560). Thus, the compound representation may be provided as input to the different second model portions (e.g., 525 or 580) to generate their respective outputs (e.g., enrichment prediction 530 or compound prediction 590).

### Training Machine Learning Models

Embodiments disclosed herein describe the training of machine learned models, such as training of a regression model and/or training of a classification model. Referring to the training of a regression model, in various embodiments, it involves using a training dataset, such as training dataset 210 shown in FIG. 2. In various embodiments, the regression model is trained to incorporate one or more covariates using supervised training techniques. Furthermore, the regression model is trained to generate an enrichment prediction (e.g., a DEL count, DEL read, or DEL index) that is a continuous value indicative of binding affinity between a compound and a target. Here, the enrichment value may be an intermediate value of the regression model that represents a de-noised and unbiased value absent effects of one or more covariates. Over training iterations, also referred to as training epochs, the regression model is trained (e.g., parameters of the regression model are adjusted) to improve its predictive capacity. The regression model can be validated or evaluated using a validation dataset, such as validation dataset 215 shown in FIG. 2.

Referring to the training of a classification model it involves using a labeled training dataset, such as labeled training dataset 220 shown in FIG. 2. In various embodiments, the classification model is trained using supervised learning to generate a binary compound prediction. Here, the binary compound prediction is an indication as to whether the compound is a predicted binder or non-binder of a target. Over training iterations, also referred to as training epochs, the classification model is trained (e.g., parameters of the classification model are adjusted) to improve its predictive capacity. The classification model can be validated or evaluated using a labeled validation dataset, such as labeled validation dataset 230 shown in FIG. 2.

In various embodiments, the training of the regression model and/or the classification model can further include one or more augmentations that selectively increase the size of the training data. For example, a compound of the training dataset (or labeled training dataset) may be represented in an initial form. In various embodiments, the compound of the training data is represented in its canonical form. Therefore, the one or more augmentations can selectively expand molecular representations of the training data to include the compound in forms that differ from its canonical form, hereafter referred to as augmented forms or augmented compound representations. Thus, by providing the regression model and/or classification model the different augmented forms of compounds during training, this further improves the ability of the regression model and/or classification model to handle different augmented forms of compounds during deployment. Examples of augmentations to generate augmented forms of compounds include, but are not limited to: enumerating tautomers of compounds, performing a transformation of compounds, wherein the transformation is any one of matched molecular pair transforms or bioisosteres, Bemis-Murcko scaffolds, node dropout, or edge dropout, generating a representation of ionization states, generating mixtures of structures associated with a tag (e.g., DNA tag), mixtures of tautomers, mixtures of conformers, mixtures of promoters, or mixtures of transformations of the one or more compounds, or generating conformers.

In various embodiments, the one or more augmentations are differently applied to different compounds of the training dataset (or labeled training dataset). Here, the one or more augmentations may be selectively applied to generate particular sets of augmented forms of the compound that differ from the initial (e.g., canonical) form of the compound. This is particularly useful because although generating a fixed set of augmentations for each compound can increase the training dataset, doing so would be highly resource intensive and costly (e.g., computationally costly and memory intensive). For example, pre-calculating a fixed set of augmented forms for every compound prior to training would require storing all the various possible augmented forms of the compound. In contrast, here, the one or more augmentations can be selectively applied to different compounds of the training dataset, thereby enabling generation of augmented forms of the compound on-the-fly without having to store pre-calculated transformations. Furthermore, after training the machine learned model using an augmented form of the compound, the augmented form can be subsequently discarded. If needed again at a subsequent time, it can be recreated on the fly from the canonical form of the compound.

In various embodiments, the one or more augmentations are differently applied to different compounds through an augmentation hyperparameter. In various embodiments, the augmentation hyperparameter controls implementation of the one or more augmentations. For example, the augmentation hyperparameter may be a tunable probability value that controls the implementation of one or more augmentations. In various embodiments, the probability value represents the probability of whether an augmentation is applied. For example, the probability value can be a value of X that is between 0 and 100. Therefore, in some scenarios (e.g., at or near X % of scenarios), an augmentation is applied to a small molecule compound. Thus, augmented forms of compounds are generated at or near X % of scenarios, and therefore, the augmented forms can be provided for training the machine learned model. Alternatively, in some scenarios (e.g., at or near 100−X % of scenarios), an augmentation is not applied to the small molecule compound. Thus, augmented forms are not generated at or near 100−X % of scenarios and therefore, the canonical forms of small molecules are provided for training the machine learned model.

In various embodiments, in the scenarios in which the augmentation hyperparameter authorizes application of an augmentation (e.g., in the X % of scenarios), a selection mechanism is implemented that determines which of the one or more augmentations are applied. In various embodiments, the selection mechanism is a random number generator. For example, the random number generator can output a random number between 1 and Z. Based on the random number output, a specific augmentation is applied. In various embodiments, Z can be a value of 2, 3, 4, 5, 6, 7, 8, 9, 10, 11, 12, 13, 14, 15, 16, 17, 18, 19, or 20. For example, assuming Z=20, there may be 20 possible augmentations that can be applied to the compound. In various embodiments, the random number generator can output multiple random numbers between 1 and Z. Therefore, for each of the random number outputs, a specific augmentation is applied. In such embodiments, multiple augmented forms of a compound can be generated.

As a specific example, a random number generator outputs a random number between 1 and 3. Here, a random number output of 1 can correspond to enumeration of a tautomer of the compound. A random number output of 2 can correspond to the generation of a representation of ionization states of the compound. A random number output of 3 can correspond to generating a conformer of the compound. Thus, assuming the random number generator outputs a random number output of 1, then tautomers of the compound are enumerated and these tautomers serve as augmented forms that can be provided for training the machine learned models.

In various embodiments, the augmentation hyperparameter may include multiple probability values that control the implementation of multiple augmentations. For example, for N different augmentations, the augmentation hyperparameter may include N probability values for controlling the implementation of the N different augmentations. For each augmentation, a random number generator is applied to output a single value. If the random number output satisfies the corresponding probability value for the augmentation, then the augmentation is applied. For example, assume 3 different augmentations and thus, 3 different probability values X, Y, and Z. The random number generator is applied for each of the augmentations to generate random output values of A, B, and C. If the random output value of A satisfies the corresponding probability value of X, then the first augmentation is applied. If the random output value of B satisfies the corresponding probability value of Y, then the second augmentation is applied. If the random output value of C satisfies the corresponding probability value of Z, then the third augmentation is applied.

In various embodiments, if the random output value of A is less than or equal to the corresponding probability value of X, then the first augmentation is applied. If the random output value of B is less than or equal to the corresponding probability value of Y, then the second augmentation is applied. If the random output value of C is less than or equal to the corresponding probability value of Z, then the third augmentation is applied.

In various embodiments, the random number outputs can correspond with particular augmentations to more heavily favor certain augmentations. For example, certain augmentations that are favored (e.g., because the machine learned models can handle favored augmented forms of the compound better than other augmented forms) can correspond to more random number outputs in comparison to less favored augmentations which would correspond to fewer random number outputs. As a specific example, a random number generator outputs a random number between 1 and 3. A random number output of 1 and 2 can both correspond to enumeration of a tautomer of the compound. A random number output of 3 can correspond to generating a conformer of the compound. In this scenario, the augmentation of enumeration of a tautomer of the compound is favored in comparison to the augmentation of generating a conformer of the compound. Thus, the enumeration of a tautomer corresponds to more random number outputs in comparison to the generation of a conformer of the compound.

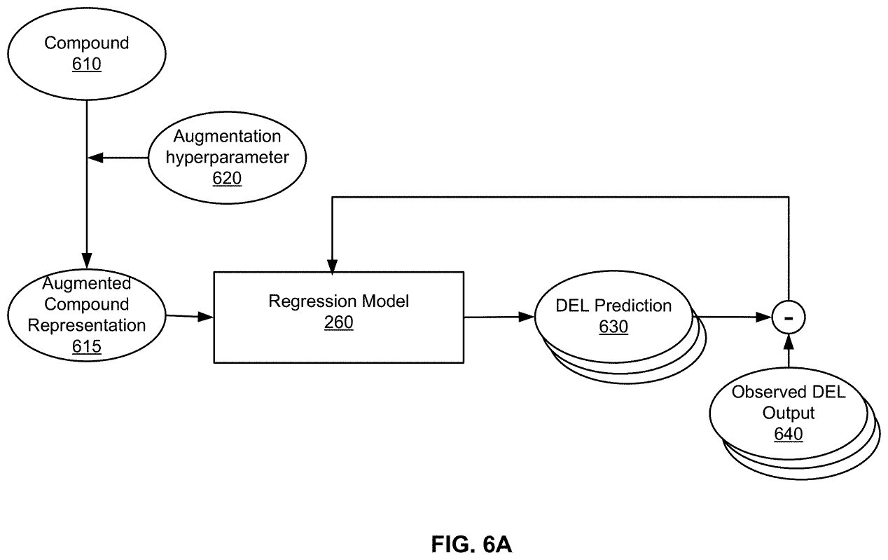

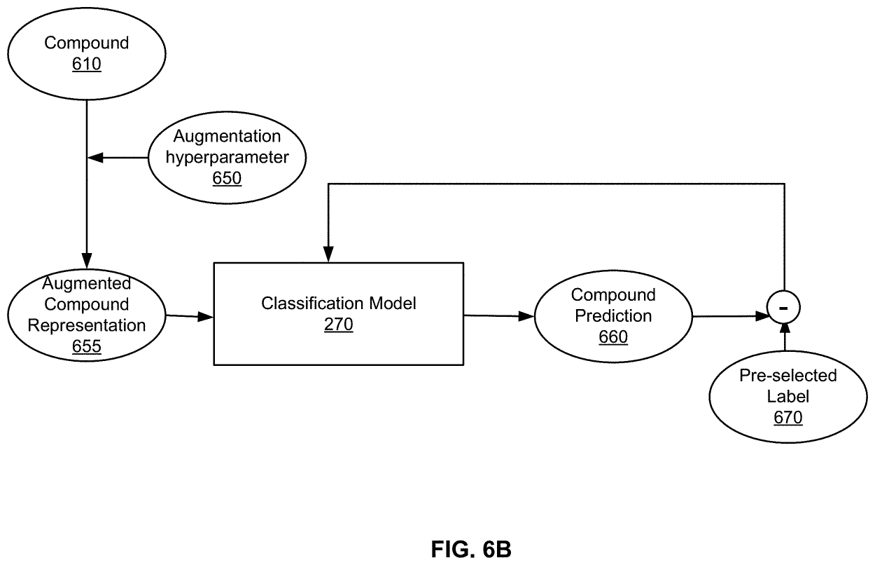

FIG. 6A depicts an example flow diagram for training a regression model, in accordance with an embodiment. FIG. 6A shows a training example that includes a compound 610 and the corresponding observed DEL output 640, such as DEL count, DEL read, or DEL index. The compound 610 may be represented in its canonical form and can undergo augmentation based on the augmentation hyperparameter 620. The augmentation hyperparameter 620 may be a tunable parameter representing a probability value that controls the implementation of the one or more augmentations. In scenarios in which the augmentation hyperparameter 620 authorizes an augmentation, a selection mechanism, such as a random number generator, can be implemented to select the augmentation to be applied. The selected augmentation is applied to generate an augmented compound representation 615, which is provided to the regression model 260. In scenarios in which the augmentation hyperparameter 620 does not authorize an augmentation, the compound 610 in its original canonical form can be provided as input to the regression model 260.

As shown in FIG. 6A, an augmented compound representation 615 is provided to the regression model 260 which generates a DEL prediction 630. In various embodiments, the regression model 260 generates two or more DEL predictions 630. Here, the DEL predictions 630 can represent DEL counts, DEL reads, or DEL indices. The DEL predictions 630 are combined with the observed DEL outputs 640. For example, differences between observed DEL outputs 640 and DEL predictions 630 are calculated. Here, the difference represents an error between the DEL prediction 630 and the observed DEL output 640. The regression model 260 is trained by back-propagating the difference between the DEL prediction 630 and the observed DEL output 640. In various embodiments, the regression model 260 is trained using a gradient based optimization technique to minimize a loss function. In various embodiments, the regression model 260 is trained using stochastic gradient descent. Examples of a loss function include any one of a mean absolute error, mean squared error, log likelihood of a negative binomial distribution, zero inflated negative binomial, or a log likelihood of a Poisson distribution.

In various embodiments, the observed DEL output 640 represents the DEL output values obtained from a DEL experiment (e.g., DEL experiment 115 shown in FIG. 1A). In various embodiments, observed DEL outputs 640 represent DEL output values (e.g., DEL output 120A and/or 120B) that are derived from experiments that modeled the effects of covariates. For example, assume that a first DEL experiment was conducted by incubating small molecule compounds with immobilized targets on beads. A second DEL experiment was conducted to model the effect of the covariate of non-specific binding to beads. Thus, the observed DEL outputs 640 can represent DEL counts (e.g., UMI counts) obtained from the first and second DEL experiments. Thus, over training epochs, the regression model 260 is trained to accurately generate DEL predictions 630, thereby enabling the modeling of the effects of covariates and binding effects.

In various embodiments, the regression model 260 can be further trained for additional augmented compound representations 615 that are generated from the compound 610. Thus, another training iteration, or training epoch, can involve providing an additional augmented compound representation 615 to the regression model 260, generating a DEL prediction, and back-propagating an error to further adjust the parameters of the regression model 260.

In various embodiments, the regression model 260 may include multiple heads or paths as described herein. At least one of the heads represents a modeled experiment which is designed to elucidate and enable incorporation of the effects of a covariate. For example, at least one of the heads generates a DEL prediction corresponding to a DEL experiment that models the effects of a covariate.

In particular embodiments, the regression model 260 includes at least two heads representing two modeled experiments that are designed to elucidate and enable incorporation of the effects of at least two covariates. For example, referring again to FIG. 5B, the second model portion 525 of the regression model 260 can include three heads, two of which model the effects of a covariate and one head models the target enrichment. As described herein, the DEL prediction can be calculated as Equation (1) or (2) described above. As shown in Equations (1) and (2), β₁, β₂ . . . βₙ₊₁ are learned parameters of the regression model, X is the target enrichment, and each of Y₁, Y₂ . . . Yₙ represents a covariate enrichment. Furthermore, the regression model can further include learned parameters α₁ . . . αₘ, examples of which are described above in Equation (3) and (4). Thus, as the regression model 260 is trained, the learned parameters β₁, β₂ . . . βₙ₊₁ and α₁ . . . αₘ are updated to improve the regression model's ability to predict the DEL prediction. As a byproduct of the regression model's ability to predict the DEL prediction, the intermediate value of the target enrichment (e.g., target enrichment 555) more accurately reflects a de-noised and unbiased value that is absent of the effects of the covariates.

FIG. 6B depicts an example flow diagram for training a classification model, in accordance with an embodiment. Generally, the classification model 270 is trained using one or more augmentations that selectively expand molecular representations of a training dataset used to train the classification model 270. FIG. 6B shows a training example that includes a compound 610 and the corresponding pre-selected label 670. The pre-selected label 670 may be a top-performing label previously selected by the dataset labeling module 140, as described herein in reference to FIG. 2.

In various embodiments, the compound 610 may be represented in its canonical form and can undergo augmentation based on the augmentation hyperparameter 650. The augmentation hyperparameter may be a tunable parameters representing a probability value that controls the implementation of the one or more augmentations. In scenarios in which the augmentation hyperparameter 650 authorizes an augmentation, a selection mechanism, such as a random number generator, can be implemented to select the augmentation to be applied. The selected augmentation is applied to generate an augmented compound representation 655, which is provided to the classification model 270. In scenarios in which the augmentation hyperparameter 650 does not authorize an augmentation, the compound 610 in its original canonical form can be provided as input to the classification model 270.

As shown in FIG. 6B, an augmented compound representation 655 is provided to the classification model 270 which generates a prediction, such as the compound prediction 660. Here, the compound prediction 660 represents a binary prediction indicative of whether the compound is likely a binder or non-binder of a target. The compound prediction 660 is combined with the pre-selected label 670. Here, the combination of the compound prediction 660 and the pre-selected label 670 represents an error between prediction of the classification model 270 and the ground truth (e.g., pre-selected label 670). The classification model 270 is trained by back-propagating the error. In various embodiments, the classification model 270 is trained using a loss function. Examples of a loss function include any one of a binary cross entropy loss, focal loss, arc loss, cosface loss, cosine based loss, or loss function based on a BEDROC metric. Therefore, for each of the training iterations (or training epochs), the parameters of the classification model 270 are updated to minimize the loss value.

In various embodiments, following training, the classification model 270 can be evaluated using a labeled validation dataset (e.g., labeled validation dataset 230 described in FIG. 2). In various embodiments, the performance of the classification model is evaluated based on a metric, which can be one or more of a Boltzmann-Enhanced Discrimination of Receiver Operating Curve (BEDROC) metric, an Area Under ROC (AUROC) metric, and an average precision (AVG-PRC) metric.

Non-Transitory Computer Readable Medium

Also provided herein is a computer readable medium comprising computer executable instructions configured to implement any of the methods described herein. In various embodiments, the computer readable medium is a non-transitory computer readable medium. In some embodiments, the computer readable medium is a part of a computer system (e.g., a memory of a computer system). The computer readable medium can comprise computer executable instructions for implementing a machine learning model for the purposes of predicting a clinical phenotype.

Computing Device

The methods described above, including the methods of training and deploying machine learning models (e.g., classification model and/or regression model), are, in some embodiments, performed on a computing device. Examples of a computing device can include a personal computer, desktop computer laptop, server computer, a computing node within a cluster, message processors, hand-held devices, multi-processor systems, microprocessor-based or programmable consumer electronics, network PCs, minicomputers, mainframe computers, mobile telephones, PDAs, tablets, pagers, routers, switches, and the like.

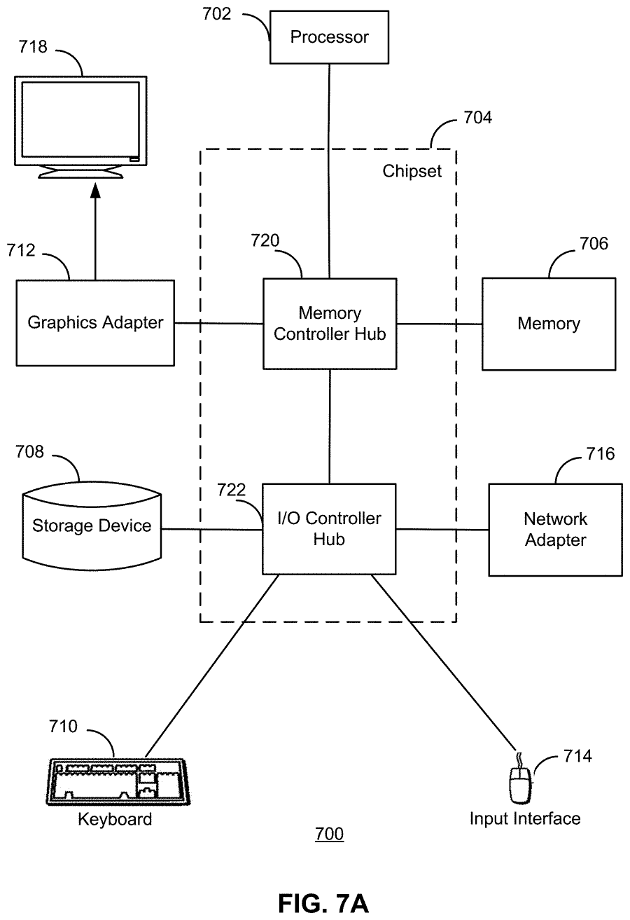

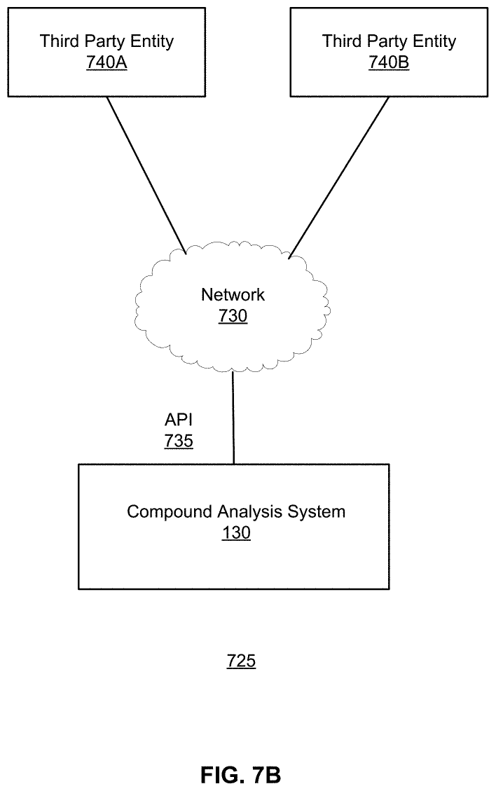

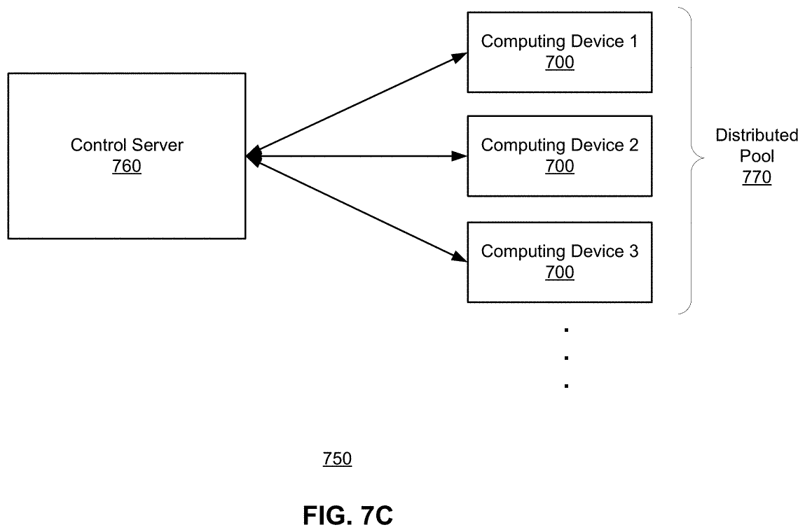

FIG. 7A illustrates an example computing device for implementing system and methods described in FIGS. 1A-1B, 2, 3A-3B, 4A-4B, 5A-5C, and 6A-6B. Furthermore, FIG. 7B depicts an overall system environment for implementing a compound analysis system, in accordance with an embodiment. FIG. 7C is an example depiction of a distributed computing system environment for implementing the system environment of FIG. 7B.

In some embodiments, the computing device 700 shown in FIG. 7A includes at least one processor 702 coupled to a chipset 704. The chipset 704 includes a memory controller hub 720 and an input/output (I/O) controller hub 722. A memory 706 and a graphics adapter 712 are coupled to the memory controller hub 720, and a display 718 is coupled to the graphics adapter 712. A storage device 708, an input interface 714, and network adapter 716 are coupled to the I/O controller hub 722. Other embodiments of the computing device 700 have different architectures.

The storage device 708 is a non-transitory computer-readable storage medium such as a hard drive, compact disk read-only memory (CD-ROM), DVD, or a solid-state memory device. The memory 706 holds instructions and data used by the processor 702. The input interface 714 is a touch-screen interface, a mouse, track ball, or other type of input interface, a keyboard, or some combination thereof, and is used to input data into the computing device 700. In some embodiments, the computing device 700 may be configured to receive input (e.g., commands) from the input interface 714 via gestures from the user. The graphics adapter 712 displays images and other information on the display 718. The network adapter 716 couples the computing device 700 to one or more computer networks.

The computing device 700 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 708, loaded into the memory 706, and executed by the processor 702.

The types of computing devices 700 can vary from the embodiments described herein. For example, the computing device 700 can lack some of the components described above, such as graphics adapters 712, input interface 714, and displays 718. In some embodiments, a computing device 700 can include a processor 702 for executing instructions stored on a memory 706.

In various embodiments, the different entities depicted in FIGS. 7A and/or FIG. 7B may implement one or more computing devices to perform the methods described above, including the methods of training and deploying one or more machine learning models (e.g., regression model and/or classification model). For example, the compound analysis system 130, third party entity 740A, and third party entity 740B may each employ one or more computing devices. As another example, one or more of the sub-systems of the compound analysis system 130 (as shown in FIG. 1B) may employ one or more computing devices to perform the methods described above.

The methods of training and deploying one or more machine learning models (e.g., regression model and/or classification model) can be implemented in hardware or software, or a combination of both. In one embodiment, a non-transitory machine-readable storage medium, such as one described above, is provided, the medium comprising a data storage material encoded with machine readable data which, when using a machine programmed with instructions for using said data, is capable of displaying any of the datasets and execution and results of a machine learning model of this invention. Such data can be used for a variety of purposes, such as patient monitoring, treatment considerations, and the like. Embodiments of the methods described above can be implemented in computer programs executing on programmable computers, comprising a processor, a data storage system (including volatile and non-volatile memory and/or storage elements), a graphics adapter, an input interface, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that is capable of recording and reproducing the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

System Environment

FIG. 7B depicts an overall system environment for implementing a compound analysis system, in accordance with an embodiment. The overall system environment 725 includes a compound analysis system 130, as described earlier in reference to FIG. 1A, and one or more third party entities 740A and 740B in communication with one another through a network 730. FIG. 7A depicts one embodiment of the overall system environment 700. In other embodiments, additional or fewer third party entities 740 in communication with the compound analysis system 130 can be included. Generally, the compound analysis system 130 implements machine learning models that make predictions, e.g., predictions for a virtual screen, hit selection and analysis, or binding affinity. The third party entities 740 communicate with the compound analysis system 130 for purposes associated with implementing the machine learning models or obtaining predictions or results from the machine learning models.

In various embodiments, the methods described above as being performed by the compound analysis system 130 can be dispersed between the compound analysis system 130 and third party entities 740. For example, a third party entity 740A or 740B can generate training data and/or train a machine learning model. The compound analysis system 130 can then deploy the machine learning model to generate predictions e.g., predictions for a virtual screen, hit selection and analysis, or binding affinity.

Third Party Entity

In various embodiments, the third party entity 740 represents a partner entity of the compound analysis system 130 that operates either upstream or downstream of the compound analysis system 130. As one example, the third party entity 740 operates upstream of the compound analysis system 130 and provide information to the compound analysis system 130 to enable the training of machine learning models. In this scenario, the compound analysis system 130 receives data, such as DEL experimental data collected by the third party entity 740. For example, the third party entity 740 may have performed the analysis concerning one or more DEL experiments (e.g., DEL experiment 115A or 115B shown in FIG. 1A) and provides the DEL experimental data of those experiments to the compound analysis system 130. Here, the third party entity 740 may synthesize the small molecule compounds of the DEL, incubate the small molecule compounds of the DEL with immobilized protein targets, eluting bound compounds, and amplifying/sequencing the DNA tags to identify putative binders. Thus, the third party entity 740 may provide the sequencing data to the compound analysis system 130.

As another example, the third party entity 740 operates downstream of the compound analysis system 130. In this scenario, the compound analysis system 130 generates predictions (e.g., predicted binders) and provides information relating to the predicted binders to the third party entity 740. The third party entity 740 can subsequently use the information identifying the predicted binders relating for their own purposes. For example, the third party entity 740 may be a drug developer. Therefore, the drug developer can synthesize the predicted binder for its investigation.

Network

This disclosure contemplates any suitable network 730 that enables connection between the compound analysis system 130 and third party entities 740. The network 730 may comprise any combination of local area and/or wide area networks, using both wired and/or wireless communication systems. In one embodiment, the network 730 uses standard communications technologies and/or protocols. For example, the network 730 includes communication links using technologies such as Ethernet, 802.11, worldwide interoperability for microwave access (WiMAX), 3G, 4G, code division multiple access (CDMA), digital subscriber line (DSL), etc. Examples of networking protocols used for communicating via the network 730 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over the network 730 may be represented using any suitable format, such as hypertext markup language (HTML) or extensible markup language (XML). In some embodiments, all or some of the communication links of the network 730 may be encrypted using any suitable technique or techniques.

Application Programming Interface (API)

In various embodiments, the compound analysis system 130 communicates with third party entities 740A or 740B through one or more application programming interfaces (API) 735. The API 735 may define the data fields, calling protocols and functionality exchanges between computing systems maintained by third party entities 740 and the compound analysis system 130. The API 735 may be implemented to define or control the parameters for data to be received or provided by a third party entity 740 and data to be received or provided by the compound analysis system 130. For instance, the API may be implemented to provide access only to information generated by one of the subsystems comprising the compound analysis system 130. The API 735 may support implementation of licensing restrictions and tracking mechanisms for information provided by compound analysis system 130 to a third party entity 740. Such licensing restrictions and tracking mechanisms supported by API 735 may be implemented using blockchain-based networks, secure ledgers and information management keys. Examples of APIs include remote APIs, web APIs, operating system APIs, or software application APIs.

An API may be provided in the form of a library that includes specifications for routines, data structures, object classes, and variables. In other cases, an API may be provided as a specification of remote calls exposed to the API consumers. An API specification may take many forms, including an international standard such as POSIX, vendor documentation such as the Microsoft Windows API, or the libraries of a programming language, e.g., Standard Template Library in C++ or Java API. In various embodiments, the compound analysis system 130 includes a set of custom API that is developed specifically for the compound analysis system 130 or the subsystems of the compound analysis system 130.

Distributed Computing Environment

In some embodiments, the methods described above, including the methods of training and implementing one or more machine learning models, are, performed in distributed computing system environments where local and remote computer systems, which are linked (either by hardwired data links, wireless data links, or by a combination of hardwired and wireless data links) through a network, both perform tasks. In some embodiments, one or more processors for implementing the methods described above may be located in a single geographic location (e.g., within a home environment, an office environment, or a server farm). In various embodiments, one or more processors for implementing the methods described above may be distributed across a number of geographic locations. In a distributed computing system environment, program modules may be located in both local and remote memory storage devices.

FIG. 7C is an example depiction of a distributed computing system environment for implementing the system environment of FIG. 7B. The distributed computing system environment 750 can include a control server 760 connected via communications network with at least one distributed pool 770 of computing resources, such as computing devices 700, examples of which are described above in reference to FIG. 7. In various embodiments, additional distributed pools 770 may exist in conjunction with the control server 760 within the distributed computing system environment 750. Computing resources can be dedicated for the exclusive use in the distributed pool 770 or shared with other pools within the distributed processing system and with other applications outside of the distributed processing system. Furthermore, the computing resources in distributed pool 770 can be allocated dynamically, with computing devices 700 added or removed from the pool 710 as necessary.

In various embodiments, the control server 760 is a software application that provides the control and monitoring of the computing devices 700 in the distributed pool 770. The control server 760 itself may be implemented on a computing device (e.g., computing device 700 described above in reference to FIG. 7A). Communications between the control server 760 and computing devices 700 in the distributed pool 770 can be facilitated through an application programming interface (API), such as a Web services API. In some embodiments, the control server 760 provides users with administration and computing resource management functions for controlling the distributed pool 770 (e.g., defining resource availability, submission, monitoring and control of tasks to performed by the computing devices 700, control timing of tasks to be completed, ranking task priorities, or storage/transmission of data resulting from completed tasks).

In various embodiments, the control server 760 identifies a computing task to be executed across the distributed computing system environment 750. The computing task can be divided into multiple work units that can be executed by the different computing devices 700 in the distributed pool 770. By dividing up and executing the computing task across the computing devices 700, the computing task can be effectively executed in parallel. This enables the completion of the task with increased performance (e.g., faster, less consumption of resources) in comparison to a non-distributed computing system environment.

In various embodiments, the computing devices 700 in the distributed pool 770 can be differently configured in order to ensure effective performance for their respective jobs. For example, a first set of computing devices 700 may be dedicated to performing collection and/or analysis of phenotypic assay data. A second set of computing devices 700 may be dedicated to performing the training of machine learning models. The first set of computing devices 700 may have less random access memory (RAM) and/or processors than the second set of second computing devices 700 given the likely need for more resources when training the machine learning models.

The computing devices 700 in the distributed pool 770 can perform, in parallel, each of their jobs and when completed, can store the results in a persistent storage and/or transmit the results back to the control server 760. The control server 760 can compile the results or, if needed, redistribute the results to the respective computing devices 700 to for continued processing.

In some embodiments, the distributed computing system environment 750 is implemented in a cloud computing environment. In this description, “cloud computing” is defined as a model for enabling on-demand network access to a shared set of configurable computing resources. For example, the control server 760 and the computing devices 700 of the distributed pool 770 may communicate through the cloud. Thus, in some embodiments, the control server 760 and computing devices 700 are located in geographically different locations. Cloud computing can be employed to offer on-demand access to the shared set of configurable computing resources. The shared set of configurable computing resources can be rapidly provisioned via virtualization and released with low management effort or service provider interaction, and then scaled accordingly. A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In this description and in the claims, a “cloud-computing environment” is an environment in which cloud computing is employed.

### EXAMPLES

### Example 1: Generating and Preparing Training Data

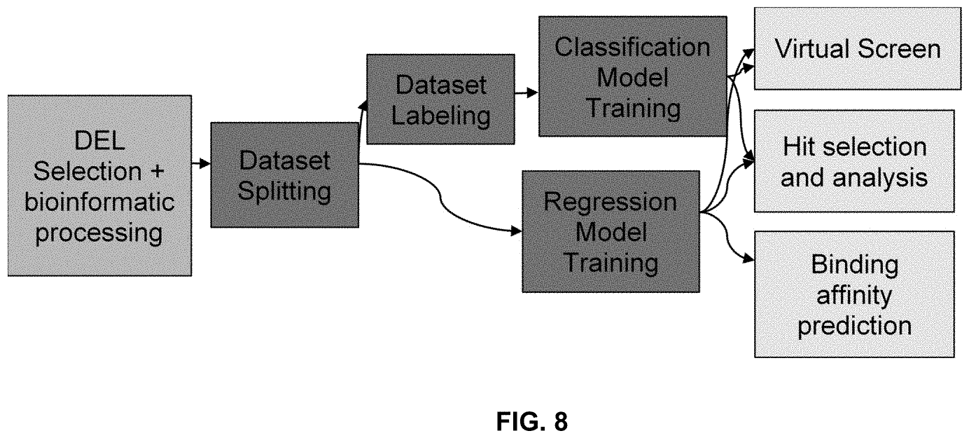

FIG. 8 shows an example flow process for conducting a virtual screen, performing hit selection and analysis, or generating binding affinity predictions. Specifically, FIG. 8 shows the first step of obtaining DEL selection data. The DEL selection data underwent dataset splitting and/or dataset labeling. Specifically, the DEL selection data that underwent dataset splitting (e.g., training dataset and validation dataset) was provided for training the regression model. Additionally, the dataset that underwent data labeling was provided for training the classification model. The trained classification model and trained regression model were then applied for the various uses cases including 1) performing a virtual screen, 2) hit selection and analysis, and 3) binding affinity prediction. In particular, the classification model can be deployed for performing a virtual screen and/or hit selection and analysis. The regression model can be deployed for each of 1) performing a virtual screen, 2) hit selection and analysis, and 3) binding affinity prediction.

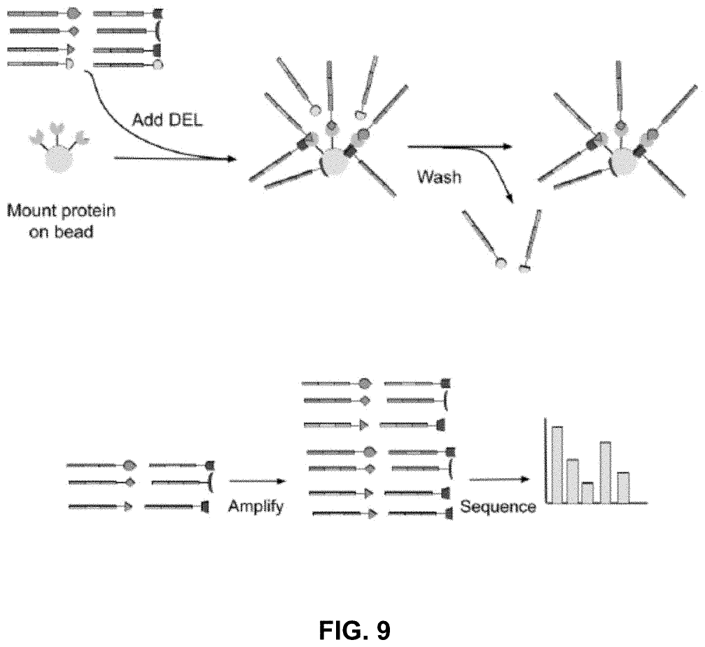

FIG. 9 shows an example diagrammatic representation of a DNA encoded library (DEL) screen. Target proteins were mounted onto beads and molecules of the DEL were added to enable binding. The mixture was washed to removed non-binders. Next, the sequences of the remaining DEL molecules undergo amplification and sequencing to identify the DEL molecules that were bound to the mounted protein. Of note, in addition to target specific binding (green) there are also multiple non target binding modes (red) that may remain with the bead mounted protein even after washing. Thus, these non-target binding modes may be erroneously sequenced even though they are not actively binders to the protein. Furthermore, amplification rates are not uniform, which leads to noisy count information. Thus, as described below and herein, machine learning models (e.g., regression models) are trained to handle appropriately handle these covariates to enable more accurate distinguishing of binders and non-binders.

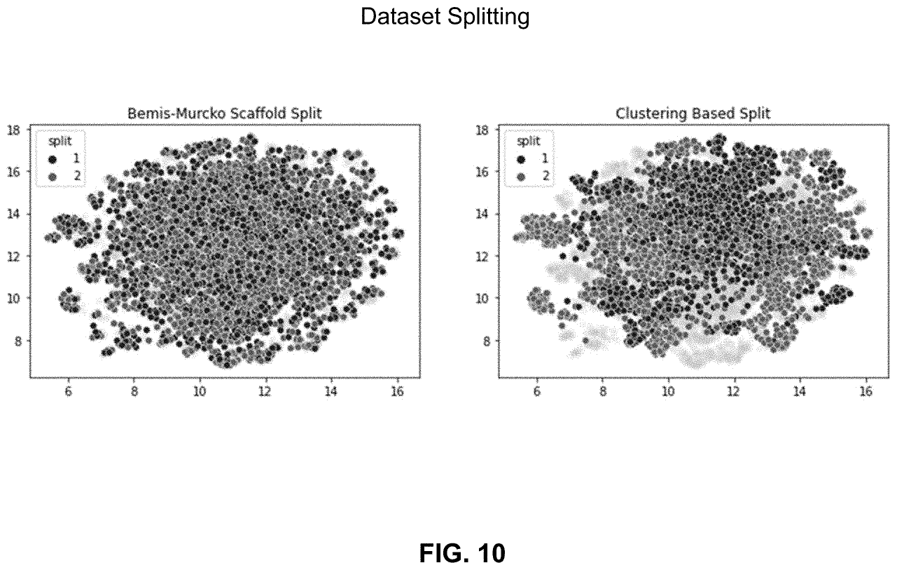

FIG. 10 depicts clustering of datasets following dataset splitting using two different methods. In particular, the left panel of FIG. 10 shows that splitting based on Bemis-Murcko scaffold shows poor separation between splits. The right panel of FIG. 10 shows the improved data splitting achieved via the methods described herein. In particular, the validation set was created that had structures different from compounds in the training set to test the model's ability to generalize to new domains. Standard methods such as random and Bemis-Murcko splitting cannot achieve the desired effects, as is depicted in the left panel of FIG. 10. This is likely due to the combinatorial nature of the library. Furthermore, many clustering methods, such as Taylor-Butina clustering, do not scale well to the hundreds of millions of compounds typically in the library. To achieve this, a representative sample of the DEL was first created by ensuring each building block in the DEL synthesis appears at least once in the sample. Next, hierarchical clustering was performed on molecular fingerprints generated from each member in the sample. The remaining members of the DEL were then assigned to the same cluster as their nearest neighbor in the initial representative sample. This procedure assigned every member of the DEL to a cluster and then these clusters were then combined together to create dataset splits. If labels for DEL members are provided, the labels can also be used to dictate assignment of clusters to dataset splits in order to create splits with balanced label proportions.

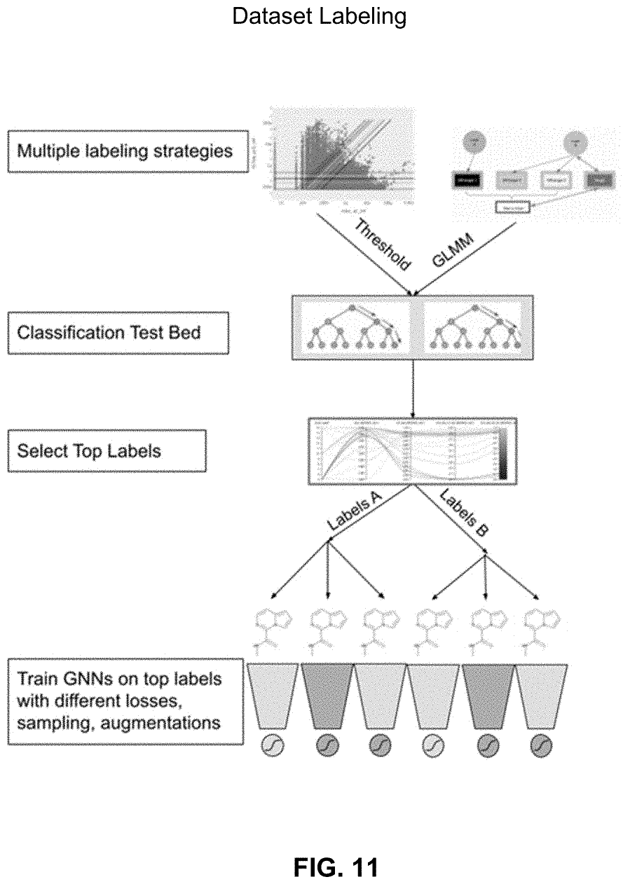

FIG. 11 depicts an example labeling scheme workflow. The dataset labeling for the classification is performed by using classical statistics such as enrichment scores over starting tag imbalance and off target signal, as well as, a generalized linear mixed model which incorporates learning of the various covariates. Many possible labeling strategies can be derived from these including various thresholds on different statistics. A test bed was created to quickly test how models performed when trained with varying label schemes and subsets of DEL data and assist selection of labeling strategy.

The test bed operates as follows: A user provides to a training function a list of chemical IDs, a list of smiles strings, and a list of labels. Optionally the user may provide a configuration tuple to specify the parameters of the fingerprint featurizer and the random forest model. Inside the training function, the provided smiles strings are converted to molecular fingerprints using rdkit and following the parameters contained in the fingerprint featurizer tuple. Additionally, multiple validation sets are loaded each with a set of smiles and labels. These too are featurized with molecular fingerprints. After featurization is complete, a balanced random forest model is trained on the user provided data with fingerprints as input and labels as target. This trained model is used to predict labels for the training and validation datasets and an array of metrics is calculated including BEDROC, ROC-AUC, and AVG-PRC. These metrics, the trained model, and a dataframe containing all predicted labels and their associated smiles strings are uploaded to weights and biases. The top performing labels are selected and used to train models (e.g., classification model and/or regression model, referred to in FIG. 11 as graph neural networks (GNNs)) with different losses, samplings, and augmentations.

### Example 2: Building Models for Virtual Screening

Multiple proprietary DEL panning datasets were screened against a challenging protein target. These datasets include control and off-target pans. Here, this example presents results for a diversity screening library of 100M compounds (Lib1) that were used for training and a separate expansion library of 2.5M compounds used for validation (Lib2).

### Classification Model

To provide a competitive baseline, a classification model was built and optimized using the same graph neural network (GNN) architecture as the regression model (GIN-E network with virtual node [15]). Binary labels were assigned for binders (positives) and non-binders (negatives) using a two-step thresholding process. First, compounds with on-target unique molecular identifier (UMI) counts below a noise threshold were discarded. Second, compound UMIs in each pan were normalized by the sum of all UMIs in the pan to yield molecular frequencies (MFs). Next, the ratio between the on-target and max control or off-target MF was calculated. If a compound's MF ratio exceeded a positive cutoff or fell below a negative cutoff, the compound was assigned a positive or negative label, respectively. Compounds with ratios falling between the cutoffs were discarded. This yielded ˜74K positives and ˜5.6M negatives. Combinations of sampling schemes and losses were experimented with to address the class imbalance, and Focal Loss [13] without balanced sampling performed best. Additionally, the model was regularized with dropout in the layers after graph readout and with input augmentations.

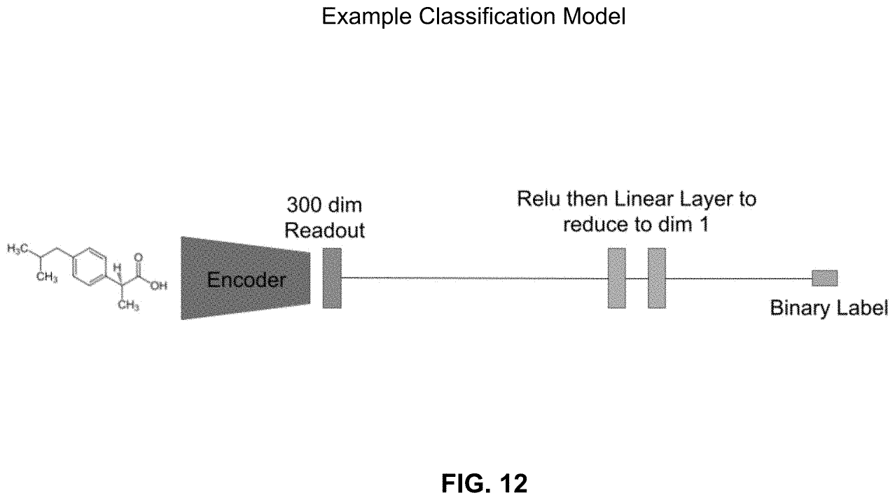

FIG. 12 depicts an example classification model. The classification model uses a GIN-E encoder and maps the encoder output to a single class prediction.

Regression Model

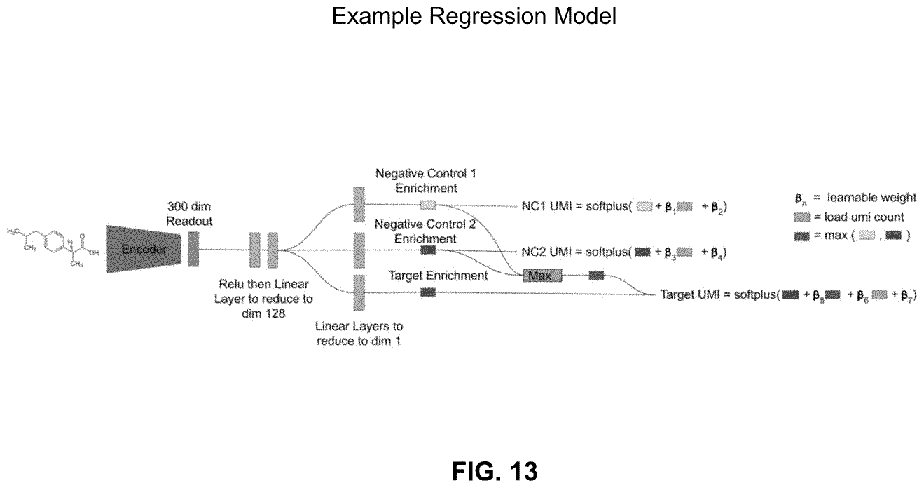

FIG. 13 depicts an example regression model. The regression model has multiple heads, each predicting an enrichment value from a reduced embedding of the encoder output. These enrichments are terms in a sum (with learned weights β) that predicts observed counts (UMI). Specifically, the regression model uses a GIN-E encoder to generate a 300 dimensional embedding. The embedding is provided to feed forward networks to further reduce the dimensionality of the embedding down to 128, and then further to a single float value (e.g., single dimension).

A negative binomial regression was used to model the UMI from each panning experiment. Here, the enrichment for each compound was modeled as the residual after accounting for various covariates such as binding to beads. As a generalization of Poisson regression, negative binomial regression incorporates a dispersion parameter α in addition to a mean variable μ. For one target pan and two no-target control pans, Cᵢ_{_{target}}˜NB(μ_{i,target}, α_{target}), C_{i,control}_{₁}˜NB(μ_{i,control}_{₁}, α_{control}_{₁}), and C_{i,control}_{₂}˜NB(μ_{i,control}_{₂}, α_{control}_{₂}) represent the UMI counts of ith compound in the respective panning experiments. Here, μᵢ was modeled as the combination of enrichment from binding to the target (R_{i,target}), enrichment from binding to the non-target media (R_{i,control}_{₁}, R_{i,control}_{₂}), and observed count of the compound in the original starting population load (Sᵢ).

μ_{i,control}_{₁}=σ(R_{i,control}_{₁}+β₁Sᵢ+β₂)

μ_{i,control}_{₂}=σ(R_{i,control}_{₂}+β₃Sᵢ+β₄)

μ_{i,target}=σ(R_{i,target}+β₅*max(R_{i,control}_{₁}R_{i,control}_{₂})+β₆Sᵢ+β₇

βᵢ are learned from the data and σ represents the softplus function, which was found to be more stable during training than the typical exponential function. The dispersion parameter, α, of the negative binomial is a single scalar, learned for each experiment. R_{i,target} and R_{i,control} was related to each compound's structure by deriving their values with a GNN operating on the compound's molecular graph. A shared encoding network generates a 128 dimensional embedding vector from atom and bond features. This embedding vector is then transformed into R_{i,target}, R_{i,control}_{₁}, and R_{i,control}_{₂} by separate feed forward networks. For these experiments, a GIN-E network with virtual node [15, 9, 10] was used for the initial encoding and two layers in each of our feed forward networks. During training, the negative log likelihood of the observed counts were summed for the target and control pans. Furthermore, the enrichment values were L2 regularized, which empirically prevented over-fitting. For a single example with count cᵢ for each panning experiment, the loss can be written as:

$$P ( {μ}_{i} , α ) = \frac{Γ ( {c}_{i} + {α}^{- 1} )}{Γ ( {c}_{i} + 1 ) Γ ( {α}^{- 1} )} {( \frac{1}{1 + {αμ}_{i}} )}^{{α}^{- 1}} {( \frac{{αμ}_{i}}{1 + {αμ}_{i}} )}^{{c}_{i}} {L}_{i , target} = - log log P ( {c}_{i , target} \vert {μ}_{i , target ,} {α}_{target} ) {L}_{i , {control}_{1}} = - log P ( {μ}_{i , {control}_{1}} , {α}_{{control}_{1}} ) {L}_{i c o n {trol}_{2}} = - log P ( {μ}_{i , {control}_{2}} , {α}_{{control}_{2}} ) {L}_{i} = {L}_{i , target} + {L}_{i , c o n {trol}_{1}} + {L}_{i , {control}_{2}} + γ {R}_{i , target}^{2} + γ {R}_{i , {control}_{1}}^{2} + γ {R}_{i , {control}_{2}}^{2}$$

where Γ(x) is the gamma function and γ is the L2 regularization rate. This negative binomial regression can be further extended with other covariates such as enrichment in other negative control pans, other target pans, compound synthesis yield, and reaction type. For this experiment, 13 negative control pans were used. During validation and inference for virtual screening, the de-noised enrichment value R_{i,target} was used to rank compounds.

### Example Implementation of Ensembled Regression Models

In the latest modeling, an extension was added for making predictions with an ensemble of regression models described above (3 different models were ensembled). For a target compound for which an inference is to be made, each model j outputted a μ_{target} and a which combined to parameterize a unique negative binomial distribution. Given three negative binomial distributions (each predicted by a model), a mixture of the three models was generated by sampling equally from each of the three distributions (e.g., 333 sampled from each distribution for a combined total of 999 samples). With these samples, any of the mixture mean, median, or nth percentile of the mixture distribution was estimated. For example, the median of the mixture distribution would be the 500th largest value. For predictions of the ensemble, the 40th percentile was used for the final virtual screening output.

### Cross Library Validation

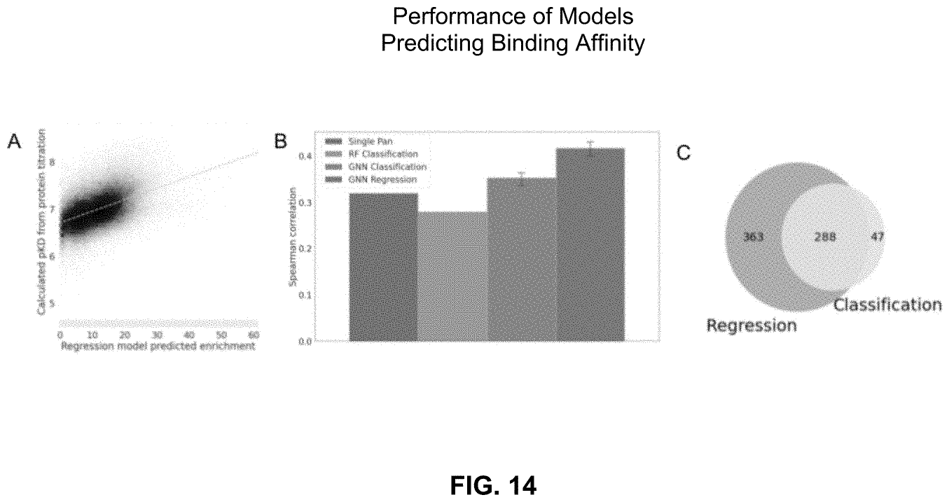

After training on Lib1, models were validated on Lib2 which had proxy binding affinity measurements. Binding affinity of a compound to a target can be measured by the equilibrium disassociation constant Kd and corresponding negative log value pKd. Lib2 was used in a set of target titration panning experiments [3] to produce titration-based pKds (t-pKds). A small portion of these t-pKds were validated with off-DNA pKd measurements (R₂=0.84). Model performance was measured by calculating the Spearman correlation coefficient between model predictions and the t-pKds. This metric aligned with the intended use of the models to rank VLs for candidate selection. The R_{target} predicted by the regression model had a 0.41 (95% CI [0.40, 0.43]) Spearman correlation with t-pKds (FIG. 14A). This exceeds both a Random Forest classification baseline (0.28) and the GNN classification model (0.35 (95% CI [0.34,0.37])) (FIG. 14B). Furthermore, both the GNN regression and GGNN classification models trained from Lib1 showed better correlation with t-pKds from Lib2 than UMI counts from a single pan of Lib2. This illustrates both the high noise in the raw UMI output from a single panning experiment and the models' ability to generalize. Finally, the two GNN models' retrieval rates were compared for strong binders in their top prediction results. The regression model had more binders in its top prediction results than the classification model (FIG. 14C).

Specifically, FIG. 14A depicts a bivariate histogram showing correlation between predicted enrichment (R_{target}) from GNN regression model and t-pKds derived from protein titration. Regression line is plotted in orange. FIG. 14B shows Spearman correlation between model predictions and t-pKds. For RF Classification and GNN Classification, predicted probability of the binder class is used for ranking. “Single Pan” represents the UMI counts from a single panning experiment done with Lib2 at the same target concentration as experiments with Lib1. Error bars on GNN Regression and Classification represents the 95% confidence interval as estimated by three model replicates. FIG. 14C depicts a Venn diagram showing number of compounds with t-pKds>=8 (n=1327) retrieved in the top 10K of the GNN Regression versus the GNN Classification model.

Virtual Screening

The regression and classification models were used to perform a virtual screen of 3.7 billion compounds from different VLs. For each model, the top 30,000 compounds were thresholded (by predicted probability of being a binder (for classification) and predicted enrichment (for regression)). This threshold roughly corresponded to the number of compounds predicted to be binders (classification) or receiving an enrichment score equivalent to the mean enrichment score of known binders in the validation set. This union of these 30,000 compound sets was clustered using Taylor-Butina clustering with a similarity cut-off of 0.25. Structural similarity was calculated via Jaccard similarity of Morgan fingerprints. For each model, 1000 compounds were selected. The selection algorithm was as follows:

For compound in list of compound sorted by rank (ascending):
If compound rank <100: select compound
Else if compound rank <200: Select compound, unless 2 compounds from cluster have already been selected
Else: Select compound, unless any compound from cluster has already been selected

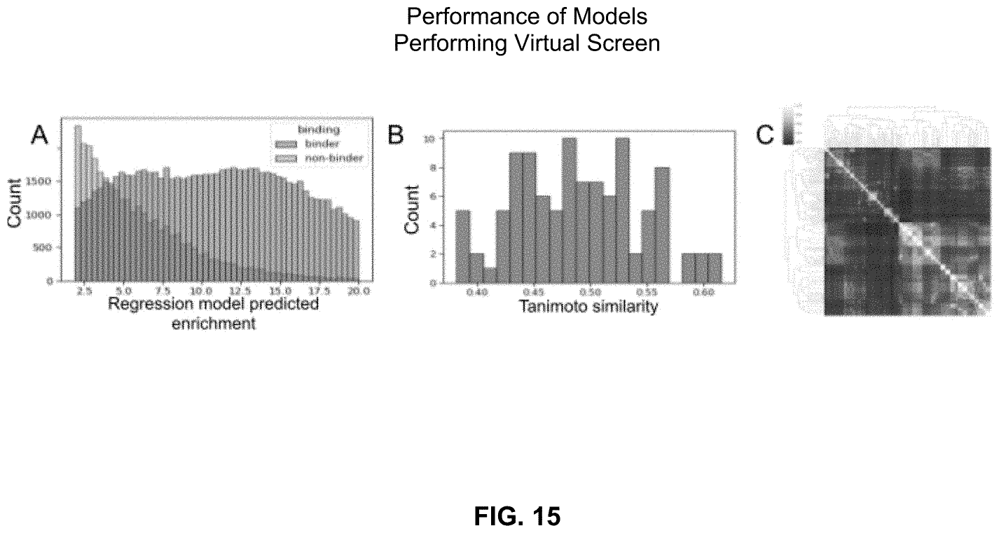

Specifically, FIG. 15 shows that regression models pick diverse compounds from the VLs. FIG. 15A shows a histogram of predicted enrichment for the overlapping region between binders and non-binders on the validation set (Lib2). FIG. 15B shows a histogram of Jaccard similarities for inference compounds with predicted enrichment >10 to their closest neighbor in the training set (Lib1). FIG. 15C is a heatmap of pairwise Jaccard similarities between compounds with predicted enrichment >10.

### CONCLUSION

DEL experiments yield datasets with low signal-to-noise ratio. In this work, a novel regression technique is implemented for modeling DEL sequencing counts that accounts for various sources of variation, such as media binding and differences in initial load. This model's predicted enrichment values have better correlation with proxy binding affinities than those of baseline classification models or experimental values from a single panning experiment. Finally, this model retrieves diverse compounds during virtual screening.

### REFERENCES

1. D. Butina. Unsupervised data base clustering based on daylight's fingerprint and tanimoto similarity: A fast and automated way to cluster small and large data sets. Journal of Chemical Information and Computer Sciences, 39(4):747-750, 07 1999.
2. M. A. Clark, R. A. Achaiya, C. C. Arico-Muendel, S. L. Belyanskaya, D. R. Benjamin, N. R. Carlson, P. A. Centrella, C. H. Chiu, S. P. Creaser, J. W. Cuozzo, C. P. Davie, Y. Ding, G. J. Franklin, K D Franzen, M. L. Gefter, S. P. Hale, N. J. V. Hansen, D. I. Israel, J. Jiang, M. J. Kavarana, M. S. Kelley, C. S. Kollmann, F Li, K. Lind, S. Mataruse, P. F. Medeiros, J. A. Messer, P. Myers, H. O'Keefe, M. C. Oliff, C. E. Rise, A. L. Satz, S. R. Skinner, J. L. Svendsen, L. Tang, K. van Vloten, R. W. Wagner, G. Yao, B. Zhao, and B. A. Morgan. Design, synthesis and selection of dna-encoded small-molecule libraries. Nature Chemical Biology, 5(9):647-654, 2009.
3. J. Cuozzo, P. Centrella, D. Gikunju, S. Habeshian, C. Hupp, A. Keefe, E. Sigel, H. Soutter, H. Thomson, Y. Zhang, and M. Clark. Discovery of a potent btk inhibitor with a novel binding mode using parallel selections with a dna-encoded chemical library. Chembiochem: a European journal of chemical biology, 18:864-871, 01 2017. doi: 10.1002/cbic.201600573.
4. W. Decurtins, M. Wichert, R. M. Franzini, F. Buller, M. A. Strays, Y. Zhang, D. Neri, and J. Scheuermann Automated screening for small organic ligands using dna-encoded chemical libraries. Nature Protocols, 11(4):764-780, 2016.
5. J. C. Faver, K. Riehle, D. R. Lancia, J. B. J. Milbank, C. S. Kollmann, N Simmons, Z. Yu, and M. M. Matzuk. Quantitative comparison of enrichment from dna-encoded chemical library selections. ACS Combinatorial Science, 21(2):75-82, 02 2019.
6. C. J. Geny, M. J. Wawer, P. A. Clemons, and S. L. Schreiber. Dna barcoding a complete matrix of stereoisomeric small molecules. Journal of the American Chemical Society, 141(26): 10225-10235, 07 2019.
7. A. Gironda-Martinez, E. J. Donckele, F. Samain, and D. Neri. Dna-encoded chemical libraries: A comprehensive review with successful stories and future challenges. ACS Pharmacology & Translational Science, 4(4):1265-1279, 08 2021.
8. C. Hafemeister and R. Satija. Normalization and variance stabilization of single-cell rna-seq data using regularized negative binomial regression. Genome Biology, 20(1):296, 2019.
9. W. Hu, B. Liu, J. Gomes, M. Zitnik, P. Liang, V. Pande, and J. Leskovec. Strategies for pre-training graph neural networks, 2020.
10. W. Hu, M. Fey, M. Zitnik, Y. Dong, H. Ren, B. Liu, M. Catasta, and J. Leskovec. Open graph benchmark: Datasets for machine learning on graphs, 2021.
11. L. Kuai, T. O'Keeffe, and C. Arico-Muendel. Randomness in dna encoded library selection data can be modeled for more reliable enrichment calculation. SLAS DISCOVERY: Advancing the Science of Drug Discovery, 23(5):405-416, 2021/09/07 2018.
12. K. S. Lim, A. G. Reidenbach, B. K. Hua, J. W. Mason, C. J. Geny, P. A. Clemons, and C. W. Coley. Machine learning on dna-encoded library count data using an uncertainty-aware probabilistic loss function, 2021.
13. T.-Y. Lin, P. Goyal, R. Girshick, K. He, and P. Dollar. Focal loss for dense object detection, 2018.
14. K. McCloskey, E. A. Sigel, S. Kearnes, L. Xue, X. Tian, D. Moccia, D. Gikunju, S. Bazzaz, B Chan, M. A. Clark, J. W. Cuozzo, M.-A. Guié, J. P. Guilinger, C. Huguet, C. D. Hupp, A. D. Keefe, C. J. Mulhern, Y. Zhang, and P. Riley. Machine learning on dna-encoded libraries: A new paradigm for hit finding. Journal of Medicinal Chemistry, 63(16):8857-8866, 08 2020. doi: 10. 1021/acs.jmedchem.0c00452. URL https://doi.org/10.1021/acsjmedchem.0c00452.
15. K. Xu, W. Hu, J. Leskovec, and S. Jegelka. How powerful are graph neural networks?, 2019.
16. Jean-Francois Truchon and Christopher I. Bayly, Evaluating Virtual Screening Methods: Good and Bad Metrics for the “Early Recognition” Problem, Journal of Chemical Information and Modeling 2007 47 (2), 488-508, doi: 10.1021/ci600426e

1-148. (canceled)

149. A method for conducting a molecular screen for a target, the method comprising:
obtaining a plurality of compounds from a library;
for each of one or more of the plurality of compounds:
applying the compound as input to one or both of:
(A) a classification model for predicting candidate compounds likely to bind to the target, wherein the classification model is trained using one or more augmentations that selectively expand molecular representations of a training dataset used to train the classification model; and
(B) a regression model trained to predict a value indicative of binding affinity between compounds and targets, wherein the regression model is trained using compounds with corresponding DNA-encoded library (DEL) outputs to incorporate two or more covariates for predicting the value indicative of binding affinity; and
selecting candidate compounds as predicted binders of the target based on one or both of the outputs of the classification model and the regression model.

150. The method of claim 149, wherein applying the compound as input comprises applying the compound as input to both the classification model and the regression model.

151. The method of claim 150, further comprising:
identifying overlapping candidate compounds predicted by the classification model and by the regression model based on the value indicative of binding affinity; and
selecting a subset of the overlapping candidate compounds as predicted binders of the target.

152. The method of claim 149, wherein applying the compound as input to a classification model for predicting candidate compounds likely to bind to the target comprises:
determining one of distance or clustering of one or more compounds within an embedding;
based on the distance or clustering of the one or more compounds within the embedding, determining whether to label the one or more compounds as candidate compounds.

153. The method of claim 149, wherein the one or more augmentations comprise:
a. enumerating tautomers of compounds during training,
b. performing a transformation of one or more compounds, wherein the transformation is any one of matched molecular pair transforms or bioisosteres, Bemis-Murcko scaffolds, node dropout, or edge dropout,
c. generating a representation of ionization states,
d. generating mixtures of structures associated with a tag, mixtures of tautomers, mixtures of conformers, mixtures of ionization states, or mixtures of transformations of the one or more compounds, or
e. generating conformers.

154. The method of claim 153, wherein the tag associated with mixtures of structures is a DNA sequence.

155. The method of claim 153, wherein the classification model comprises a tunable hyperparameter that controls implementation of the one or more augmentations.

156. The method of claim 155, wherein the tunable hyperparameter is a probability value that controls the implementation of the one or more augmentations.

157. The method of claim 156, wherein the one or more augmentations are further selected for implementation using a random number generator.

158. The method of claim 149, wherein the regression model comprises a first portion that analyzes the compound and outputs a fixed dimensional embedding.

159. The method of claim 158, wherein applying the compound as input to the regression model trained to predict a value indicative of binding affinity comprises:
using the embedding to generate an enrichment value representing the value indicative of binding affinity.

160. The method of claim 159, wherein using the embedding to generate the enrichment value comprises providing the embedding as input to a feed forward network, wherein the feed forward network generates the enrichment value for a modeled experiment.

161. The method of claim 159, wherein the enrichment value represents an intermediate value within the regression model.

162. The method of claim 161, wherein the regression model is further trained to predict one or more DEL predictions that model one or more experiments, wherein at least one of the one or more DEL predictions is generated using at least the intermediate value of the enrichment value.

163. The method of claim 159, wherein applying the compound as input to the regression model trained to predict a value indicative of binding affinity further comprises:
using the embedding to generate one or more covariate enrichment values that correspond to one or more negative control experiments.

164. The method of claim 163, wherein the negative control experiment models effects of the covariate across a set of proteins or for a binding site.

165. The method of claim 164, wherein the binding site is a target binding site or an orthogonal binding site.

166. The method of claim 149, wherein each of the two or more covariates are any of non-specific binding via controls and other targets data, starting tag imbalance, experimental conditions, chemical reaction yields, side and truncated products, errors from the library synthesis, DNA affinity to target, sequencing depth, and sequencing noise such as PCR bias.

167. A method for predicting binding affinity between a compound and a target, the method comprising:
obtaining the compound;
applying the compound as input to a regression model trained to predict a value indicative of binding affinity between compounds and targets,
wherein the regression model is trained using compounds with corresponding DNA-encoded library (DEL) outputs to incorporate two or more covariates for predicting the value indicative of binding affinity.

168. A non-transitory computer readable medium for conducting a molecular screen for a target, the non-transitory computer readable medium comprising instructions that, when executed by a processor, cause the processor to:
obtain a plurality of compounds from a library;
for each of one or more of the plurality of compounds:
apply the compound as input to one or both of:
(A) a classification model for predicting candidate compounds likely to bind to the target, wherein the classification model is trained using one or more augmentations that selectively expand molecular representations of a training dataset used to train the classification model; and
(B) a regression model trained to predict a value indicative of binding affinity between compounds and targets, wherein the regression model is trained using compounds with corresponding DNA-encoded library (DEL) outputs to incorporate two or more covariates for predicting the value indicative of binding affinity; and
select candidate compounds as predicted binders of the target based on one or both of the outputs of the classification model and the regression model.
